(19) United States
(12) Patent Application Publication
(10) Pub. No.: US 2008/0221200 A1
(43) Pub. Date: Sep. 11, 2008

(54) Combination of Organic Compounds

(76) Inventors: Malcolm Allison, Basel (CH); Bryan Burkey, Winchester, MA (US); Thomas Edward Hughes, Concord, MA (US); Daniel Matthew Kemp, Melrose, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/067,822

(22) PCT Filed: Sep. 28, 2006
(86) PCT No.: PCT/US06/38203

§ 371 Date: Mar. 24, 2008

Related U.S. Application Data
(60) Provisional application No. 60/722,629, filed on Sep. 30, 2005.

(51) Int. Cl. A61K 31/402; A61P 3/10; A61P 17/06; A61P 37/06

(52) U.S. Cl. 514/423

(57) ABSTRACT

The present invention relates to a combination, such as a combined preparation or pharmaceutical composition, respectively, comprising of a DPP IV inhibitor or a pharmaceutically acceptable salt thereof and comprising at least one immunosuppressive or immunomodulator agent, or a pharmaceutically acceptable salt thereof.

The present invention furthermore relates to the use of such a combination for the prevention, delay of progression or treatment of diseases and disorders that may be inhibited by DPP IV inhibition, for the prevention, delay of progression or treatment of autoimmune diseases, and the disorders associated therewith, or for the prevention, delay of progression or treatment of graft rejection.

This invention relates to a pharmaceutical combination comprising a DPP-IV inhibitor and at least an immunosuppressive or immunomodulator agent, in particular for the prevention, delay of progression or treatment of diseases and disorders that may be inhibited by DPP IV inhibition, for the prevention, delay of progression or treatment of autoimmune diseases, e.g. type 1 diabetes and the disorders associated therewith, or graft rejection.

In spite of numerous treatment options for organ transplant and autoimmune disease patients, there remains a need for effective and safe immunosuppressive agents and a need for their preferential use in combination therapy.

Type 1 diabetes is also called insulin dependent diabetes mellitus (IDDM). In type 1 diabetes, the insulin-producing pancreatic beta-cells undergo an autoimmune attack by the body itself, and the pancreas is rendered incapable of making insulin. Europe has one of the highest prevalences with more than one million patients. The disease has devastating vascular complications with staggering costs; present treatment modalities are far from optimal, neither cure nor prevention being available. Individuals diagnosed with type 1 diabetes require regular insulin injections to survive. Islet cell transplants and pancreas transplants can in some cases eliminate the need for insulin injections. However, these procedures still require the patient to stay on a life-long schedule of anti rejection medications.

Type 1 diabetes is characterized by a progressive loss of pancreatic beta cells due to an unfavourable balance between the destructive autoimmune processes targeting beta cells on the one hand and the regenerative capacity of these cells on the other hand. This imbalance eventually leads to total loss of beta cells and endogenous insulin secretion.

However, shortly after insulin treatment is initiated in newly diagnosed patients with type 1 diabetes a paradoxical improvement occurs: the patient's residual beta-cell capacity in creases and a remission period (“honeymoon”) follows in which the patient's need for exogenous insulin treatment is lowered and in some cases even totally abolished and metabolic control is near to optimal. Consequently the remission period may reflect a period of relative beta-cell recovery or the clinical presentation of an injured but still regenerating beta cell mass. Accordingly, the duration of the remission period will be proportional to the regeneration potential of the beta cells. It is therefore possible that an increase in the beta cell mass in newly diagnosed patients with type 1 diabetes may relieve the stress on the remaining beta cells and thus protect them against autoimmune destruction.

The principle of pharmacological intervention to preserve beta-cell function in the remission period has previously been demonstrated with diazoxide as an adjunct therapy to regular insulin regimen in islet cell antibody (ICA)-positive adults patients with newly diagnosed type 1 diabetes [Bjork E, et al Diabetes (1996) 45:1427-30 and Bjork E, et al Diabetes Care (1998) 21:427-430) but due to intolerable side effects (lowering of blood pressure, edema, increased hair growth) treatment with this compound is not amendable to normal clinical praxis. Therefore at present there is no basis for pharmacological intervention to pre serve beta-cell function in the remission period on a regular basis.

Type 1 diabetes is a chronic autoimmune disease in which insulin-producing cells (P cells) within the pancreatic islets of Langerhan are selectively targeted and destroyed by an infiltrate of immunological cells. This infiltrate causes an inflammatory affect on the islets, known as insulitis. Treatment protocols after onset of Type 1 diabetes are particularly problematic, since by the time diabetes is diagnosed in humans, insulitis has already progressed dramatically, resulting in a cell loss of more than 80%. Islet transplantation is a potentially successful treatment for Type 1 diabetes, although severe P cell destruction is required to warrant such a procedure.

There is a need for early stage therapies for inhibition of insulitis and other conditions of islet dysfunction. Protocols which could begin prior to disease onset in individuals at risk would be particularly beneficial. Significant progress has been made in identifying risk factors in individuals susceptible to developing Type 1 diabetes. However, the above noted conventional treatment protocols for Type 1 diabetes are not practical as preventative therapies due to expense and undesirable side-effects. Insulitis is a prediabetic stage, which usually precedes onset of Type 1 diabetes, and thus there is a need for prophylactic protocols for inhibition of insulitis, which could result ultimately in delay or prevention of Type 1 diabetes.

Type 2 diabetes usually develops in people older than 40 or people who are overweight (obese). In general, the treatment of type 2 diabetes patients does not involve insulin therapy but modification of certain lifestyle aspects (e.g. exercise, weight loss, a strict diet) and sometimes oral antidiabetics is sufficient to control blood glucose levels.

Type 1 diabetes is frequently diagnosed in childhood, and is sometimes referred to as juvenile diabetes for that reason. Early diagnosis is important to prevent some of the more serious complications of diabetes, which include heart disease, blindness, high blood pressure, nerve damage, and kidney failure. However, although type 1 diabetes tends to Occur most frequently in young, lean individuals, usually before 30 years of age, older patients do also present this form of diabetes. This type of type 1 diabetes is usually referred to as latent autoimmune diabetes of adulthood (LADA). Like the more common juvenile type 1 diabetes, LADA is caused by immune-mediated destruction of the insulin-producing pancreatic beta cells. LADA is also known as slow-onset type 1 diabetes, late-onset autoimmune diabetes of adulthood, and type 1 diabetes. The main difference between juvenile type 1 diabetes and LADA is the age of diagnosis—generally thirty years or older. A Methods for the diagnosis of LADA are e.g. described in the patent application WO2005054512 A2.

Thus, type 1 diabetes can be present at any age, and the factors that determine the age of clinical manifestation are not known. Type 2 diabetes (Adult-Onset Diabetes Mellitus) generally occurs when the body develops insulin resistance a result of genetic factors and obesity, and is typically diagnosed in adulthood. Insulin resistance causes hyperglycemia and, because of prolonged demand for insulin production, deterioration of the pancreatic beta cells. The combination of insulin resistance and decreased beta cell function ultimately causes type 2 diabetes.

LADA is often misdiagnosed as type 2 diabetes because of the late age of onset. Like other forms of type I diabetes, patients with LADA require insulin injections to normalize their blood glucose levels. To assume that obese patients over 30 years are inevitably type 2 diabetes cases is inappropriate, and can lead to incorrect treatment. Therefore, when diagnosing diabetes, it is very important to be able to reliably discriminate between type 1 diabetes and type 2 diabetes. Known diagnostic methods include a blood test for the presence (in type 1 diabetes patients) of islet cell antibodies (ICA), insulin auto antibodies (IAA), and/or glutamic acid decarboxylase (a beta cell protein known as (IAD) which can confirm a LADA diagnosis. The amount of c-peptide present, a protein that is generated during insulin production, can also be used to differentiate type 1 diabetes or LADA from type 2 diabetes.

It has now been found that a combination comprising at least one immunosuppressive or immunomodulator agent e.g., as defined below, and a DPP-IV inhibitor as co-agent, e.g., as defined below, has a beneficial effect on autoimmune diseases, e.g. type I diabetes and the disorders associated therewith, or graft rejection or conditions/disorders that might be treated by DPP-IV inhibition.

Thus, the present invention relates to combinations, such as a combined preparation or pharmaceutical composition, respectively, comprising;
i) a DPP IV inhibitor or a pharmaceutically acceptable salt thereof, and
ii) at least one active ingredient selected from an immunosuppressive or an immunomodulator agent, or a pharmaceutically acceptable salt thereof.

Preferably the present invention relates to a combination (pharmaceutical combination), such as a combined preparation or pharmaceutical composition, respectively, comprising;
i) a DPP IV inhibitor or a pharmaceutically acceptable salt thereof, and
ii) at least one active ingredient selected from an immunosuppressive or an immunomodulator agent, or a pharmaceutically acceptable salt thereof,
   and at least one additional pharmaceutically acceptable carrier.

Preferably the combination is a pharmaceutical composition or a combined pharmaceutical preparation.

In this pharmaceutical composition, the combination partners (i) and (ii) can be administered together, one after the other or separately in one combined unit dosage form or in two separate unit dosage forms. The unit dosage form may also be a fixed combination.

The term “at least one therapeutic agent” shall mean that in addition to the DPP IV inhibitor one or more, for example two, furthermore three, active ingredients as specified according to the present invention can be combined. Preferably one or two agents selected from an immunosuppressive and/or an immunomodulator.

The term “DPP-IV” as used herein is intended to mean dipeptidyl peptidase IV, also known as CD26. DPP-IV, a serine protease belonging to the group of post-proline/alanine cleaving amino-dipeptidases, specifically removes the two N-terminal amino acids from proteins having proline or alanine in position 2. DPP-IV can be used in the control of glucose metabolism because its substrates include the insulinotropic hormones glucagon like peptide-1 (GLP-1) and gastric inhibitory peptide (GIP). GLP-1 and GIP are active only in their intact forms; removal of their two N-terminal amino acids inactivates them.

In vivo administration of synthetic inhibitors of DPP-IV prevents N-terminal degradation of GLP-1 and GIP, resulting in higher plasma concentrations of these hormones, increased insulin secretion and, therefore, improved glucose tolerance.

The term “DPP-IV inhibitor” is intended to indicate a molecule that exhibits inhibition of the enzymatic activity of DPP-IV and functionally related enzymes, such as from 1-100% or 20-80% inhibition, and specially preserves the action of substrate molecules, including but not limited to GLP-1, GIP, peptide histidine methionine, substance P, neuropeptide Y, and other molecules typically containing alanine or proline residues in the second amino terminal position. Treatment with DPP-IV inhibitors prolongs the duration of action of peptide substrates and increases levels of their intact, undegraded forms leading to a spectrum of biological activities relevant to the disclosed invention.

For that purpose, chemical compounds are tested for their ability to inhibit the enzyme activity of purified CD26/DPP-IV. Briefly, the activity of CD26/DPP-IV is measured in vitro by its ability to cleave the synthetic substrate Gly-Pro-p-nitroanilide (Gly-Pro-pNA). Cleavage of Gly-Pro-pNA by DPP-IV liberates the product p-nitroanilide (pNA), whose rate of appearance is directly proportional to the enzyme activity. Inhibition of the enzyme activity by specific enzyme inhibitors slows down the generation of pNA. Stronger interaction between an inhibitor and the enzyme results in a slower rate of generation of pNA. Thus, the degree of inhibition of the rate of accumulation of pNA is a direct measure of the strength of enzyme inhibition. The accumulation of pNA is measured spectrophotometrically. The inhibition constant, Ki, for each compound is determined by incubating fixed amounts of enzyme with several different concentrations of inhibitor and substrate.

In the present context “a DPP-IV inhibitor” is also intended to comprise active metabolites and prodrugs thereof, such as active metabolites and prodrugs of DPP-IV inhibitors. An active “metabolite” is an active derivative of a DPP-IV inhibitor produced when the DPP-IV inhibitor is metabolized. A “prodrug” is a compound that is either metabolized to a DPP-IV inhibitor or is metabolized to the same metabolite(s) as a DPP-IV inhibitor.

DPP-IV inhibitors are known in the art. For example, DPP-IV inhibitors are in each case generically and specifically disclosed e.g. in WO 98/19998, DE19616 486 A1, WO 00/34241, WO 95/15309, WO 01/72290, WO01/52825, WO 9310127, WO 9925719, WO 9938501, WO 9946272, WO 9967278 and WO 9967279.

Preferred DPP-IV inhibitors are described in the following patent applications; WO 02053548 especially compounds 1001 to 1293 and examples 1 to 124, WO 02067918 especially compounds 1000 to 1278 and 2001 to 2159, WO 02066627 especially the described examples, WO 02/068420 especially all the compounds specifically listed in the examples I to LXIII and the described corresponding analogues, even preferred compounds are 2(28), 2(88), 2(119), 2(136) described in the table reporting IC50, WO 02083128 especially examples 1 to 13, US 2003096846 especially the specifically described compounds, WO 2004/037181 especially examples 1 to 33, WO 0168603 especially compounds of examples 1 to 109, EP1258480 especially compounds of examples 1 to 60, WO 0181337 especially examples 1 to 118, WO 02083109 especially examples 1A to 1D, WO 030003250 especially compounds of examples 1 to 166, most preferably 1 to 8, WO 03035067 especially the compounds described in the examples, WO 03/035057 especially the compounds described in the examples, US2003216450 especially examples 1 to 450, WO 99/46272 especially compounds of claims 12, 14, 15 and 17, WO 0197808 especially compounds of claim 2, WO 03002553 especially compounds of examples 1 to 33, WO 01/34594 especially the compounds described in the examples 1 to 4, WO 02051836 especially examples 1 to 712, EP1245568 especially examples 1 to 7, EP1258476 especially examples 1 to 32, US 2003087950 especially the described examples, WO 02/076450 especially examples 1 to 128, WO 03000180 especially examples 1 to 162, WO 03000181 especially examples 1 to 66, WO 03004498 especially examples 1 to 33, WO 0302942 especially examples 1 to 68, U.S. Pat. No. 6,482,844 especially the described examples, WO 0155105 especially the compounds listed in the examples 1 and 2, WO 0202560 especially examples 1 to 166, WO 03004496 especially examples 1 to 103, WO 03/024965 especially examples 1 to 54, WO 0303727 especially examples 1 to 209, WO 0368757 especially examples 1 to 88, WO 03074500 especially examples 1 to 72, examples 4.1 to 4.23, examples 5.1 to 5.10, examples 6.1 to 6.30, examples 7.1 to 7.23, examples 8.1 to 8.10, examples 9.1 to 9.30, WO 02038541 especially examples 1 to 53, WO 02062764 especially examples 1 to 293, preferably the compound of example 95 (2-{{3-(Aminomethyl)-4-butoxy-2-neopentyl-1-oxo-1,2 dihydro-6-isoquinolinyl}oxy}acetamide hydrochloride), WO 02308090 especially examples 1-1 to 1-109, examples 2-1 to 2-9, example 3, examples 4-1 to 4-19, examples 5-1 to 5-39, examples 6-1 to 6-4, examples 7-1 to 7-10, examples 8-1 to 8-8, examples 7-1 to 7-7 of page 90, examples 8-1 to 8-59 of pages 91 to 95, examples 9-1 to 9-33, examples 10-1 to 10-20, US 2003225102 especially compounds 1 to 115, compounds of examples 1 to 121, preferably compounds a) to z), aa) to az), ba) to bz), ca) to cz) and da) to dk), WO 0214271 especially examples 1 to 320 and US 2003096857 and WO 2004/052850 especially the specifically described compounds such as examples 1 to 42 and compounds of claim 1, DE 102 56 264 A1 especially the described compounds such as examples 1 to 181 and the compounds of claim 5, WO 04/076433 especially the compounds specifically described, such as listed in table A, preferably the compounds listed in table B, preferably compounds I to XXXXVII, or compounds of claims 6 to 49, WO 04/071454 especially the specifically described compounds e.g. compounds 1 to 53 or compounds of tables Ia to If, or compounds of claims 2 to 55, WO 02/068420 especially the compounds specifically described, such as the compounds I to LXIII or Beispiele I and analogues 1 to 140 or Beispiele 2 and analogues 1 to 174 or Beispiele 3 and analogues 1, or Beispiele 4 to 5, or Beispiele 6 and analogues 1 to 5, or Beispiele 7 and analogues 1-3, or Beispiele 8 and analogue 1, or Beispiele 9, or Beispiele 10 and analogues 1 to 531 even preferred are compounds of claim 13, WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965024942 especially the compounds specifically described, such compounds 1 to 54, Wo03002593 especially the compounds specifically described, such compounds table 1 or of claims 2 to 15, WO03037327 especially the compounds specifically described, such compounds of examples 1 to 209 WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965024942 especially the compounds specifically described, such compounds 1 to 54, Wo03002593 especially the compounds specifically described, such compounds table 1 or of claims 2 to 15, WO03037327 especially the compounds specifically described, such compounds of examples 1 to 209, WO0238541, WO0230890. WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965 especially the compounds specifically described, such compounds 1 to 54, WO 03002593 especially the compounds specifically described, such compounds table 1 or of claims 2 to 15, WO03037327 especially the compounds specifically described, such compounds of examples 1 to 209, WO0238541 especially the compounds specifically described, such compounds of examples 1 to 53, WO 03/002531 especially the compounds specifically described preferably the compounds listed on page 9 to 13, most preferably the compounds of examples 1 to 46 and even preferred compound of example 9, U.S. Pat. No. 6,395,767 preferably compound of examples 1 to 109 most preferably compound of example 60, U.S. application Ser. No. 09/788,173 filed Feb. 16, 2001 (attorney file LA50) especially the described examples, WO99/38501 especially the described examples, WO99/46272 especially the described examples and DE1 9616 486 A1 especally val-pyr, val-thiazolidide, isoleucyl-thiazolidide, isoleucyl-pyrrolidide, and fumar salts of isoleucyl-thiazolidide and isoleucyl-pyrrolidide.

Further preferred DPP-IV inhibitors include the specific examples disclosed in U.S. Pat. No. 6,124,305 and U.S. Pat. No. 6,107,317, International Patent Applications, Publication Numbers WO 95153 09 and WO 9818763.

In each case in particular in the compound claims and the final products of the working examples, the subject matter of the final products, the pharmaceutical preparations and the claims are hereby incorporated into the present application by reference to these publications.

Published patent application WO 9819998 discloses N—(N′-substituted glycyl)-2-cyano pyrrolidines, in particular 1-[2-[5-Cyanopyridin-2-yl]amino]-ethylamino]acetyl-2-cyano-(S)-pyrrolidine (NVP-DPP728).

Published patent application WO 0034241 and published patent US 6110949 disclose N-substituted adamantyl-amino-acetyl-2-cyano pyrrolidines and W (substituted glycyl)-4-cyano pyrrolidines respectively. DPP-IV inhibitors of interest are specially those cited in claims 1 to 4. In particular these applications describe the compound I-[[(3-Hydroxy-1-adamantyl)amino]acetyl]-2-cyano-(S)-pyrrolidine (also known as LAF237 or vildagliptin).

Published patent application WO 9515309 discloses amino acid 2-cyanopyrrolidine amides as inhibitors of DPP-IV Published patent application WO 9529691 discloses peptidyl derivates of diesters of alpha-aminoalkylphosphonic acids, particularly those with proline or related structures. DPP-IV inhibitors of interest are specially those cited in Table 1 to 8.

In WO 01/72290 DPP-IV inhibitors of interest are specially those cited in example 1 and claims 1, 4, and 6.

WO01/52825 specially discloses (S)-1-{2-[5-cyanopyridin-2-yl)amino]ethyl-aminoacetyl)-2-cyano-pyrrolidine or (S)-1-[(3-hydroxy-1-adamantyl)amino]acetyl-2-cyano-pyrrolidine.

Published patent application WO 9310127 discloses proline boronic esters useful as DPP-IV inhibitors. DPP-IV inhibitors of interest are specially those cited in examples 1 to 19.

Published patent application WO 9925719 discloses sulphostin, a DPP-IV inhibitor prepared by culturing a Streptomyces microorganism.

Published patent application WO 9938501 discloses N-substituted 4-8 membered heterocyclic rings. DPP-IV inhibitors of interest are specially those cited in claims 15 to 20.

Published patent application WO 9946272 discloses phosphoric compounds as inhibitors of DPP-IV. DPP-IV inhibitors of interest are specially those cited in claims 1 to 23.

Published patent applications WO 9967278 and WO 9967279 disclose DPP-IV prodrugs and inhibitors of the form A-B-C where C is either a stable or unstable inhibitor of DPP-IV.

Other preferred DPP-IV inhibitors are the compounds of formula I, II or III disclosed in the patent application WO 03/057200 on page 14 to 27. Most preferred DPP-IV inhibitors are the compounds specifically described on pages 28 and 29.

Any of the substances disclosed in the above mentioned patent documents, hereby included by reference, are considered potentially useful as DPP-IV inhibitors to be used in carrying out the present invention.

In a further preferred embodiment, the DPP-IV inhibitor is a N-peptidyl-O-aroyl hydroxylamine or a pharmaceutically acceptable salt thereof. Aroyl is, for example, naphthylcarbonyl; or benzoyl which is unsubstituted or mono- or disubstituted, for example, by lower alkoxy, lower alkyl, halogen or, preferably, nitro. The peptidyl moiety comprises preferably two α-amino acids, e.g. glycine, alanine, leucine, phenylalanine, lysine or proline, of which the one attached directly to the hydroxylamine nitrogen atom is preferably proline.

Preferably, the N-peptidyl-O-aroyl hydroxylamine is a compound of formula VII

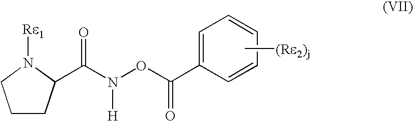

wherein
j is 0, 1 or 2;
Rε₁ represents the side chain of a natural amino acid; and
Rε₂ represents lower alkoxy, lower alkyl, halogen or nitro;
or a pharmaceutically acceptable salt thereof.

In a very preferred embodiment of the invention, the N-peptidyl-O-aroyl hydroxylamine is a compound of formula VIIIa

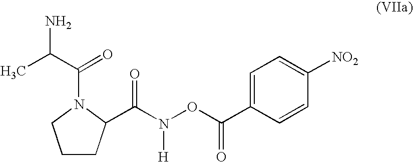

or a pharmaceutically acceptable salt thereof.

N-Peptidyl-O-aroyl hydroxylamines, e.g. of formula VII or VIIa, and their preparation are described by H. U. Demuth et al. in J. Enzyme Inhibition 1988, Vol. 2, pages 129-142, especially on pages 130-132.

Preferred DPP-IV inhibitors are N-substituted adamantyl-amino-acetyl-2-cyano pyrrolidines, N (substituted glycyl)-4-cyano pyrrolidines, N—(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl)amino]ethylamino]acetyl-2-cyano-(S)-pyrrolidine and pharmaceutical salts thereof.

Preferred DPP-IV inhibitors are those described by Mona Patel and col. (Expert Opinion Investig Drugs. 2003 April; 12(4):623-33) on the paragraph 5, especially P32/98, K-364, FE-999011, BDPX, NVP-DDP-728 and others, which publication is hereby incorporated by reference especially the described DPP-IV inhibitors.

Another preferred inhibitor is the compound BMS-477118 disclosed in WO 2001068603 or U.S. Pat. No. 6,395,767 (compound of example 60) also known as is (1S,3S,5S)-2-[(2S)-2-amino-2-(3-hydroxytricyclo[3.3.1.1^{3,7}]dec-1-yl)-1-oxoethyl]-2-azabicyclo[3.1.0]hexane-3-carbonitrile, benzoate (1:1) as depicted in Formula M of the patent application WO 2004/052850 on page 2, and the corresponding free base, (1S,3S,5S)-2-[(2S)-2-amino-2-(3-hydroxy-tricyclo[3.3.1.1^{3,7}]dec-1-yl)-1-oxoethyl]-2-azabicyclo-[3.1.0]hexane-3-carbonitrile (M′) and its monohydrate (M″) as depicted in Formula M of the patent application WO 2004/052850 on page 3. The compound BMS-477118 is also known as saxagliptin.

Another preferred inhibitor is the compound GSK23A disclosed in WO 03/002531 (example 9) also known as (2S,4S)-1-((2R)-2-Amino-3-[(4-methoxybenzyl)sulfonyl]-3-methylbutanoyl)-4-fluoropyrrolidine-2-carbonitrile hydrochloride.

FE-999011 is described in the patent application WO 95/15309 page 14, as compound No. 18.

P32/98 or P3298 (CAS number: 251572-86-8) also known as 3-[(2S,3S)-2-amino-3-methyl-1-oxopentyl]thiazolidine can be used as 3-[(2S,3S)-2-amino-3-methyl-1-oxopentyl]thiazolidine and (2E)-2-butenedioate (2:1) mixture such as shown below

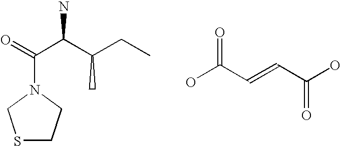

and is described in WO 99/61431 and also in Diabetes 1998, 47, 1253-1258, in the name of Probiodrug, as well as the compound P93/01 described by the same company.

Other very preferred DPP-IV inhibitors of the invention are described in the International patent application WO 02/076450 (especially the examples 1 to 128) and by Wallace T. Ashton (Bioorganic & Medicinal Chemistry Letters 14 (2004) 859-863) especially the compound 1 and the compounds listed in the tables 1 and 2. The preferred compound is the compound 21e (table 1) of formula:

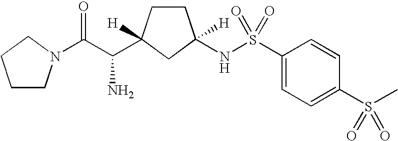

Other preferred DPP-IV inhibitors are described in the patent applications WO 2004/037169 especially those described in the examples 1 to 48 and WO 02/062764 especially the described examples 1 to 293, even preferred are the compounds 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide described on page 7 and also in the patent application WO2004/024184 especially in the reference examples 1 to 4.

Other preferred DPP-IV inhibitors are described in the patent application WO 03/004498 especially examples 1 to 33 and most preferably the compound of the formula

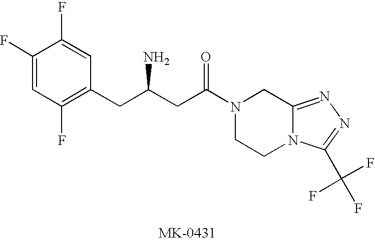

described by the example 7 and also known as MK-0431 or Sitagliptin.

Preferred DPP-IV inhibitors are also described in the patent application WO 2004/037181 especially examples 1 to 33 and most preferably the compounds described in the claims 3 to 5.

Preferred DPP-IV inhibitors are N-substituted adamantyl-amino-acetyl-2-cyano pyrrolidines, N (substituted glycyl)-4-cyano pyrrolidines, N—(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl)amino]ethylamino]acetyl-2-cyano-(S)-pyrrolidine, MK-431 and pharmaceutical salts thereof.

Most preferred DPP-IV inhibitors are selected from [S]-1-[2-(5-cyano-2-pyridinylamino)ethylamino]acetyl-2-pyrrolidine carbonitrile monohydrochloride, (S)-1-[(3-hydroxy-1-adamantyl)amino]acetyl-2-cyano-pyrrolidine and L-threo-isoleucyl thiazolidine (compound code according to Probiodrug: P32/98 as described above), MK-0431, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally pharmaceutical salts thereof.

Especially preferred are 1-{2-[(5-cyanopyridin-2-yl)amino]ethylamino}acetyl-2 (S)-cyano-pyrrolidine dihydrochloride (DPP728) (also named [S]-1-[2-(5-cyano-2-pyridinylamino)ethylamino]acetyl-2-pyrrolidine carbonitrile monohydrochloride), of formula

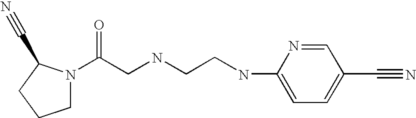

especially the dihydrochloride and monohydrochloride thereof,
and 1-[(3-hydroxy-1-adamantyl)amino]acetyl-2-cyano-, (S) (also named (S)-1-[(3-hydroxy-1-adamantyl)amino]acetyl-2-cyano-pyrrolidine, LAF237 or vildagliptin) of formula

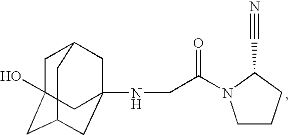

and L-threo-isoleucyl thiazolidine (compound code according to Probiodrug: P32/98 as described above), MK-0431, GSK23A, saxagliptin, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally pharmaceutical salts thereof.

DPP728 and vildagliptin are specifically disclosed in Example 3 of WO 98/19998 and Example 1 of WO 00/34241, respectively. The DPP-IV inhibitor P32/98 (see above) is specifically described in Diabetes 1998, 47, 1253-1258. DPP728 and LAF237 can be formulated as described on page 20 of WO 98/19998 or in WO 00/34241 or in the International Patent Application No. EP2005/000400 (application number).

Especially preferred are orally active DPP-IV inhibitors.

Any of the substances disclosed in the above mentioned patent documents or scientific publications, hereby included by reference, are considered potentially useful as DPP-IV inhibitors to be used in carrying out the present invention.

In each case in particular in the compound claims and the final products of the working examples, the subject matter of the final products, the pharmaceutical preparations and the claims are hereby incorporated into the present application by reference to these publications.

The term immunosuppressive drugs comprises e.g. a calcineurin inhibitor, e.g. cyclosporin A, FK 506 or ISATX247; a mTOR inhibitor, e.g. rapamycin, 40-O-(2-hydroxyethyl)-rapamycin, CC1779, ABT578, AP23573, AP23464, AP23675, AP23841, TAFA-93, biolimus 7 or biolimus 9; an ascomycin having immuno-suppressive properties, e.g. ABT-281, ASM981, etc.; corticosteroids; cyclophosphamide; azathioprene; methotrexate; a S1P receptor agonist e.g. FTY720 or an analogue thereof; leflunomide; mizoribine; mycophenolic acid or a salt or ester thereof, e.g. mycophenolate sodium or mycophenolate mofetil; 15-deoxyspergualine or an immunosuppressive homologue, analogue or derivative thereof; immunosuppressive monoclonal antibodies, e.g. monoclonal antibodies to leukocyte receptors, e.g. MHC, CD2, CD3, CD4, CD 11a/CD18, CD7, CD25, CD 27, B7, CD40, CD45, CD58, CD 137, ICOS, CD150 (SLAM), OX40, 4-1 BB or their ligands, e.g. CD154″.

Calcineurin inhibitors include e.g. cyclosporins and FK506.

Cyclosporins to which the present invention applies are any of those having pharmaceutical utility, e.g. as immunosuppressive agents, as known and described in the art, in particular Cyclosporin A (also known as Ciclosporin), Cyclosporin G, [O-(2-hydroxyethyl)-(D)Ser]⁸-Ciclosporin, and [3′-dehydroxy-3′-keto-MeBmt]¹-[Val]²-Ciclosporin. Cyclosporin A is preferred.

“Cyclosporin A” may be used in the form of a microemulsion concentrate, e.g. as disclosed in U.S. Pat. No. 5,342,625, U.S. Pat. No. 5,741,512, U.S. Pat. No. 5,866,159, U.S. Pat. No. 5,916,589, U.S. Pat. No. 5,962,014, U.S. Pat. No. 5,962,017, U.S. Pat. No. 6,024,978, U.S. Pat. No. 6,007,840, or in the form of soft gel capsules, e.g. as disclosed in EP649651, or in the form of a hydrosol, e.g. as disclosed in U.S. Pat. No. 5,389,382, the contents thereof being incorporated herein by reference. Preferably Cys A is administered (or used) in the form as commercially available under the Tradename Neoral or Sandimmun Neoral.

FK-506, also known as tacrolimus, is a macrolide lactone produced from the fermentation broth of Streptomyces tsukubaensis and has e.g. been described in Journal of Antibiotics 1987, 40: p. 1249-1255 and in Journal of Antibiotics 1987, 40: p. 1256-1265.

A mTOR inhibitor is a compound which targets intracellular mTOR (“mammalian Target Of Rapamycin”). mTOR is a family member of phosphatidylinositol 3-kinase (PI3-kinase) related kinase. Rapamycin and rapamycin derivatives inhibit the mTOR pathway via a complex with its intracellular receptor FKBP12 (FK506-binding protein 12).

Rapamycin is a known macrolide antibiotic produced by Streptomyces hygroscopicus. By rapamycin derivative is meant a substituted rapamycin having mTOR inhibiting properties, e.g. rapamycin substituted in position 40 and/or 16 and/or 32, for example a compound of formula I

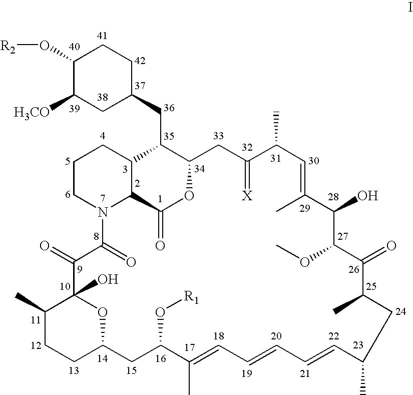

wherein
R₁ is CH₃ or C₃₋₆alkynyl,
R₂ is H, —CH₂—CH₂—OH, 3-hydroxy-2-(hydroxymethyl)-2-methyl-propanoyl or tetrazolyl, and
X is ═O, (H,H) or (H,OH)
   provided that R₂ is other than H when X is ═O and R₁ is CH₃,
   or a prodrug thereof when R₂ is —CH₂—CH₂—OH, e.g. a physiologically hydrolysable ether thereof.

Representative rapamycin derivatives of formula I are e.g. 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxymethyl)-2-methylpropanoate]-rapamycin (also called CC1779) or 40-epi-(tetrazolyl)-rapamycin (also called ABT578). A preferred compound is e.g. 40-0-(2-hydroxyethyl)-rapamycin (everolimus) disclosed in Example 8 in WO 94/09010 (referred hereinafter as Compound A), or 32-deoxorapamycin or 16-pent-2-ynyloxy-32(S)-dihydro-rapamycin as disclosed in WO 96/41807.

Rapamycin derivatives may also include the so-called rapalogs, e.g. as disclosed in WO 98/02441 and WO01/14387 and WO0364383, e.g. AP23573, AP23464, AP23675 or AP23841.

Further examples of a rapamycin derivative are those disclosed under the name TAFA-93, biolimus-7 or biolimus-9.

The term immunomodulatory compounds comprises e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y″.

Preferably the immunomodulator is a S1P receptor agonist or modulator.

S1P receptor agonists or modulators are compounds which signal as agonists at one or more sphingosine-1 phosphate receptors, e.g. S1P1 to S1P8. Agonist binding to a S1P receptor may e.g. result in dissociation of intracellular heterotrimeric G-proteins into Gα-GTP and Gβγ-GTP, and/or increased phosphorylation of the agonist-occupied receptor and activation of downstream signaling pathways/kinases.

The binding affinity of S1P receptor agonists or modulators to individual human S1P receptors may be determined in following assay:

S1P receptor agonist or modulator activities of compounds are tested on the human S1P receptors S1P₁, S1P₃, S1P₂, S1P₄ and S1P₅. Functional receptor activation is assessed by quantifying compound induced GTP [γ-³⁵S] binding to membrane protein prepared from transfected CHO or RH7777 cells stably expressing the appropriate human S1P receptor. The assay technology used is SPA (scintillation proximity based assay). Briefly, DMSO dissolved compounds are serially diluted and added to SPA-bead (Amersham-Pharmacia) immobilised S1P receptor expressing membrane protein (10-20 μg/well) in the presence of 50 mM Hepes, 100 mM NaCl, 10 mM MgCl₂, 10 μM GDP, 0.1% fat free BSA and 0.2 nM GTP [γ-³⁵S] (1200 Ci/mmol). After incubation in 96 well microtiterplates at RT for 120 min, unbound GTP [γ-³⁵S] is separated by a centrifugation step. Luminescence of SPA beads triggered by membrane bound GTP [γ-³⁵S] is quantified with a TOPcount plate reader (Packard). EC₅₀s are calculated using standard curve fitting software. In this assay, the S1P receptor agonists preferably have a binding affinity to S1P receptor <50 nM.

Preferred S1P receptor agonists or modulators are e.g. compounds which in addition to their S1P binding properties also have accelerating lymphocyte homing properties, e.g. compounds which elicit a lymphopenia resulting from a re-distribution, preferably reversible, of lymphocytes from circulation to secondary lymphatic tissue, without evoking a generalized immunosuppression. Naïve cells are sequestered; CD4 and CD8 T-cells and B-cells from the blood are stimulated to migrate into lymph nodes (LN) and Peyer's patches (PP).

The lymphocyte homing property may be measured in following Blood Lymphocyte Depletion assay:

A S1P receptor agonist or modulator or the vehicle is administered orally by gavage to rats. Tail blood for hematological monitoring is obtained on day −1 to give the baseline individual values, and at 2, 6, 24, 48 and 72 hours after application. In this assay, the S1P receptor agonist or modulator depletes peripheral blood lymphocytes, e.g. by 50%, when administered at a dose of e.g. <20 mg/kg.

Examples of appropriate S1P receptor agonists or modulators are, for example
Compounds as disclosed in EP627406A1, e.g. a compound of formula I

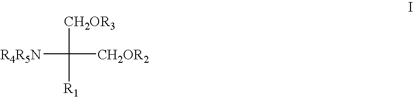

wherein R₁ is a straight- or branched (C₁₂₋₂₂)chain
which may have in the chain a bond or a hetero atom selected from a double bond, a triple bond, O, S, NR₆, wherein R₆ is H, C₁₋₄alkyl, aryl-C₁₋₄alkyl, acyl or (C₁₋₄alkoxy)carbonyl, and carbonyl, and/or
   which may have as a substituent C₁₋₄alkoxy, C₂₋₄alkenyloxy, C₂₋₄alkynyloxy, arylC₁₋₄alkyl-oxy, acyl, C₁₋₄alkylamino, C₁₋₄alkylthio, acylamino, (C₁₋₄alkoxy)carbonyl, (C₁₋₄alkoxy)-carbonylamino, acyloxy, (C₁₋₄alkyl)carbamoyl, nitro, halogen, amino, hydroxyimino, hydroxy or carboxy; or

### R₁ is

a phenylalkyl wherein alkyl is a straight- or branched (C₆₋₂₀)carbon chain; or
a phenylalkyl wherein alkyl is a straight- or branched (C₁₋₃₀)carbon chain wherein said phenylalkyl is substituted by
a straight- or branched (C₆₋₂₀)carbon chain optionally substituted by halogen,
a straight- or branched (C₆₋₂₀)alkoxy chain optionally substituted by halogen,
a straight- or branched (C₆₋₂₀)alkenyloxy,
phenyl-C₁₋₁₄alkoxy, halophenyl-C₁₋₄alkoxy, phenyl-C₁₋₁₄alkoxy-C₁₋₁₄alkyl, phenoxy-C₁₋₄alkoxy or phenoxy-C₁₋₄alkyl,
cycloalkylalkyl substituted by C₆₋₂₀alkyl,
heteroarylalkyl substituted by C₆₋₂₀alkyl,
heterocyclic C₆₋₂₀alkyl or
heterocyclic alkyl substituted by C₂₋₂₀alkyl,
   and wherein
   the alkyl moiety may have
in the carbon chain, a bond or a heteroatom selected from a double bond, a triple bond, O, S, sulfinyl, sulfonyl, or NR₆, wherein R₆ is as defined above, and
as a substituent C₁₋₄alkoxy, C₂₋₄alkenyloxy, C₂₋₄alkynyloxy, arylC₁₋₄alkyloxy, acyl, C₁₋₄alkyl-amino, C₁₋₄alkylthio, acylamino, (C₁₋₄alkoxy)carbonyl, (C₁₋₄alkoxy)carbonylamino, acyloxy, (C₁₋₄alkyl)carbamoyl, nitro, halogen, amino, hydroxy or carboxy, and
   each of R₂, R₃, R₄ and R₅, independently, is H, C₁₋₄ alkyl or acyl
   or a pharmaceutically acceptable salt or hydrate thereof;
Compounds as disclosed in EP 1002792A1, e.g. a compound of formula II

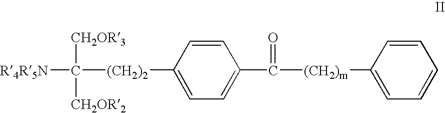

wherein m is 1 to 9 and each of R′₂, R′₃, R′₄ and R′₅, independently, is H, C₁₋₆alkyl or acyl,
or a pharmaceutically acceptable salt or hydrate thereof;

Compounds as disclosed in EP0778263 A1, e.g. a compound of formula III

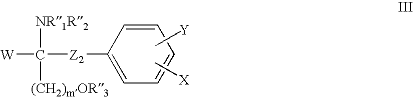

wherein W is H; C₁₋₆alkyl, C₂₋₆alkenyl or C₂₋₆alkynyl; unsubstituted or by OH substituted phenyl; R″₄O(CH₂)ₙ; or C₁₋₆alkyl substituted by 1 to 3 substituents selected from the group consisting of halogen, C₃₋₈cycloalkyl, phenyl and phenyl substituted by OH;
X is H or unsubstituted or substituted straight chain alkyl having a number p of carbon atoms or unsubstituted or substituted straight chain alkoxy having a number (p−1) of carbon atoms, e.g. substituted by 1 to 3 substitutents selected from the group consisting of C₁₋₆alkyl, OH, C₁₋₆alkoxy, acyloxy, amino, C₁₋₆alkylamino, acylamino, oxo, haloC₁₋₆alkyl, halogen, unsubstituted phenyl and phenyl substituted by 1 to 3 substituents selected from the group consisting of C₁₋₆alkyl, OH, C₁₋₆alkoxy, acyl, acyloxy, amino, C₁₋₆alkylamino, acylamino, haloC₁₋₆alkyl and halogen; Y is H, C₁₋₆alkyl, OH, C₁₋₆alkoxy, acyl, acyloxy, amino, C₁₋₆alkylamino, acylamino, haloC₁₋₆alkyl or halogen, Z₂ is a single bond or a straight chain alkylene having a number or carbon atoms of q,
each of p and q, independently, is an integer of 1 to 20, with the proviso of 6≦p+q≦23, m′ is 1, 2 or 3, n is 2 or 3,
each of R″₁, R″₂, R″₃ and R″₄, independently, is H, C₁₋₄alkyl or acyl,
or a pharmaceutically acceptable salt or hydrate thereof,
Compounds as disclosed in WO02/18395, e.g. a compound of formula IVa or IVb

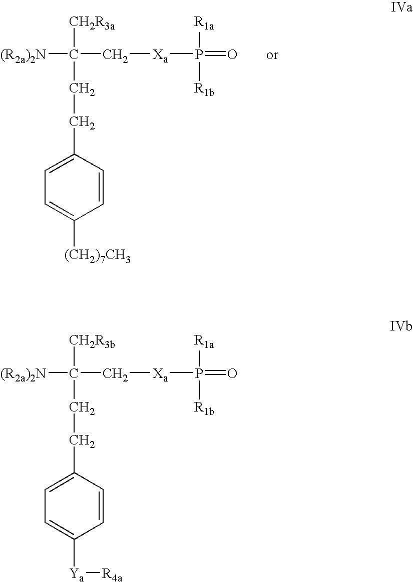

wherein Xₐ is O, S, NR₁ₛ or a group —(CH₂)ₙₐ—, which group is unsubstituted or substituted by 1 to 4 halogen; nₐ is 1 or 2, R₁ₛ is H or (C₁₋₄)alkyl, which alkyl is unsubstituted or substituted by halogen; R₁ₐ is H, OH, (C₁₋₄)alkyl or O(C₁₋₄)alkyl wherein alkyl is unsubstituted or substituted by 1 to 3 halogen; R_{1b} is H, OH or (C₁₋₄)alkyl, wherein alkyl is unsubstituted or substituted by halogen; each R₂ₐ is independently selected from H or (C₁₋₄)alkyl, which alkyl is unsubstituted or substituted by halogen; R₃ₐ is H, OH, halogen or O(C₁₋₄)alkyl wherein alkyl is unsubstituted or substituted by halogen; and R_{3b} is H, OH, halogen, (C₁₋₄)alkyl wherein alkyl is unsubstituted or substituted by hydroxy, or O(C₁₋₄)alkyl wherein alkyl is unsubstituted or substituted by halogen; Yₐ is —CH₂—, —C(O)—, —CH(OH)—, —C(═NOH)—, O or S, and R₄ₐ is (C₄₋₁₄)alkyl or (C₄₋₁₄)alkenyl;
or a pharmaceutically acceptable salt or hydrate thereof;
Compounds as disclosed in WO 02/076995, e.g. a compound of formula V

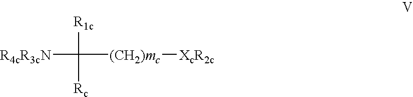

wherein
m_{c} is 1, 2 or 3;
X_{c} is O or a direct bond;
R_{1c} is H; C₁₋₆alkyl optionally substituted by OH, acyl, halogen, C₃₋₁₀cycloalkyl, phenyl or hydroxy-phenylene; C₂₋₆alkenyl; C₂₋₆alkynyl; or phenyl optionally substituted by OH;
R_{2c} is

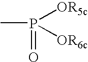

wherein R_{5c} is H or C₁₋₄alkyl optionally substituted by 1, 2 or 3 halogen atoms, and R_{6c} is H or C₁₋₄alkyl optionally substituted by halogen;
   each of R_{3c} and R_{4c}, independently, is H, C₁₋₄alkyl optionally substituted by halogen, or acyl, and
R_{c} is C₁₃₋₂₀alkyl which may optionally have in the chain an oxygen atom and which may optionally be substituted by nitro, halogen, amino, hydroxy or carboxy; or a residue of formula (a)

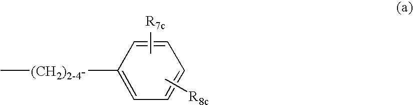

wherein R_{7c} is H, C₁₋₄alkyl or C₁₋₄alkoxy, and R₈ₛ is substituted C₁₋₂₀alkanoyl, phenylC₁₋₁₄alkyl wherein the C₁₋₁₄alkyl is optionally substituted by halogen or OH, cycloalkylC₁₋₁₄alkoxy or phenylC₁₋₁₄alkoxy wherein the cycloalkyl or phenyl ring is optionally substituted by halogen, C₁₋₄alkyl and/or C₁₋₄alkoxy, phenylC₁₋₁₄alkoxyC₁₋₁₄alkyl, phenoxyC₁₋₁₄alkoxy or phenoxyC₁₋₁₄alkyl,
R_{c} being also a residue of formula (a) wherein R_{8c} is C₁₋₁₄alkoxy when R_{1c} is C₁₋₄alkyl, C₂₋₆alkenyl or C₂₋₆alkynyl,
   or a compound of formula VI

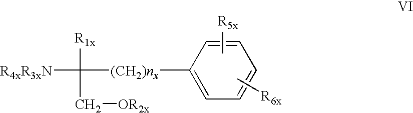

wherein
nₓ is 2, 3 or 4
R₁ₓ is H; C₁₋₆alkyl optionally substituted by OH, acyl, halogen, cycloalkyl, phenyl or hydroxy-phenylene; C₂₋₆alkenyl; C₂₋₆alkynyl; or phenyl optionally substituted by OH;
R₂ₓ is H, C₁₋₄ alkyl or acyl
each of R₃ₓ and R₄ₓ, independently is H, C₁₋₄alkyl optionally substituted by halogen or acyl,
R₅ₓ is H, C₁₋₄alkyl or C₁₋₄alkoxy, and
R₆ₓ is C₁₋₂₀ alkanoyl substituted by cycloalkyl; cyloalkylC₁₋₁₄alkoxy wherein the cycloalkyl ring is optionally substituted by halogen, C₁₋₄alkyl and/or C₁₋₄alkoxy; phenylC₁₋₁₄alkoxy wherein the phenyl ring is optionally substituted by halogen, C₁₋₄alkyl and/or C₁₋₄alkoxy,
R₆ₓ being also C₄₋₁₄alkoxy when R₁ₓ is C₂₋₄alkyl substituted by OH, or pentyloxy or hexyloxy when R₁ₓ is C₁₋₄alkyl,
   provided that R₆ₓ is other than phenyl-butylenoxy when either R₅ₓ is H or R₁ₓ is methyl,
   or a pharmaceutically acceptable salt or hydrate thereof;
   Compounds as disclosed in WO02/06268A1, e.g. a compound of formula VII

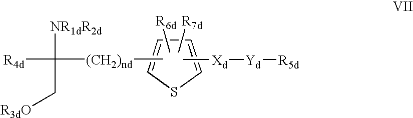

wherein each of R_{1d} and R_{2d}, independently, is H or an amino-protecting group;
R_{3d} is hydrogen, a hydroxy-protecting group or a residue of formula

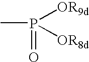

R_{4d} is C₁₋₄alkyl;
n_{d} is an integer of 1 to 6;
X_{d} is ethylene, vinylene, ethynylene, a group having a formula—D-CH₂— (wherein D is carbonyl, —CH(OH)—, O, S or N), aryl or aryl substituted by up to three substitutents selected from group a as defined hereinafter;
Y_{d} is single bond, C₁₋₁₀alkylene, C₁₋₁₀alkylene which is substituted by up to three substitutents selected from groups a and b, C₁₋₁₀alkylene having O or S in the middle or end of the carbon chain, or C₁₋₁₀alkylene having O or S in the middle or end of the carbon chain which is substituted by up to three substituents selected from groups a and b;
R_{5d} is hydrogen, C₃₋₆cycloalkyl, aryl, heterocyclic group, C₃₋₆cycloalkyl substituted by up to three substituents selected from groups a and b, aryl substituted by up to three substituents selected from groups a and b, or heterocyclic group substituted by up to three substituents selected from groups a and b;
each of R_{6d} and R_{7d}, independently, is H or a substituent selected from group a;
each of R_{8d} and R_{9d}, independently, is H or C₁₋₄alkyl optionally substituted by halogen;
<group a> is halogen, lower alkyl, halogeno lower alkyl, lower alkoxy, lower alkylthio, carboxyl, lower alkoxycarbonyl, hydroxy, lower aliphatic acyl, amino, mono-lower alkylamino, di-C₁₋₄alkylamino, acylamino, cyano or nitro; and
<group b> is C₃₋₆cycloalkyl, aryl or heterocyclic group, each being optionally substituted by up to three substituents selected from group a;
with the proviso that when R_{5d} is hydrogen, Y_{d} is a either a single bond or linear C₁₋₁₀ alkylene, or a pharmacologically acceptable salt, ester or hydrate thereof;
Compounds as disclosed in JP-14316985 (JP2002316985), e.g. a compound of formula VIII

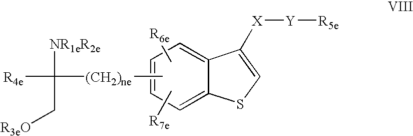

wherein R₁ₑ, R₂ₑ, R₃ₑ, R₄ₑ, R₅ₑ, R₆ₑ, R₇ₑ, nₑ, Xₑ and Yₑ are as disclosed in JP-14316985;
or a pharmacologically acceptable salt, ester or hydrate thereof;
Compounds as disclosed in WO 03/29184 and WO 03/29205, e.g. compounds of formula IX

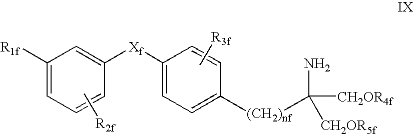

wherein X_{f} is O, S, SO or SO₂
R_{1f} is halogen, trihalomethyl, OH, C₁₋₇alkyl, C₁₋₄alkoxy, trifluoromethoxy, phenoxy, cyclohexylmethyloxy, pyridylmethoxy, cinnamyloxy, naphthylmethoxy, phenoxymethyl, CH₂—OH, CH₂—CH₂—OH, C₁₋₄alkylthio, C₁₋₄alkylsulfinyl, C₁₋₄alkylsulfonyl, benzylthio, acetyl, nitro or cyano, or phenyl, phenylC₁₋₄alkyl or phenyl-C₁₋₄alkoxy each phenyl group thereof being optionally substituted by halogen, CF₃, C₁₋₄alkyl or C₁₋₄alkoxy;
R_{2f} is H, halogen, trihalomethyl, C₁₋₄alkoxy, C₁₋₇alkyl, phenethyl or benzyloxy;
R_{3f} H, halogen, CF₃, OH, C₁₋₇alkyl, C₁₋₄alkoxy, benzyloxy or C₁₋₄alkoxymethyl;
each of R_{4f} and R_{5f}, independently is H or a residue of formula

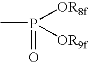

wherein each of R_{8f} and R_{9f}, independently, is H or C₁₋₄alkyl optionally substituted by halogen; and
n_{f} is an integer from 1 to 4;
e.g. 2-amino-2-[4-(3-benzyloxyphenoxy)-2-chlorophenyl]propyl-1,3-propane-diol or 2-amino-2-[4-(benzyloxyphenylthio)-2-chlorophenyl]propyl-1,3-propane-diol, or a pharmacological salt or hydrate thereof;
Compounds as disclosed in WO03/062252A1, e.g. a compound of formula X

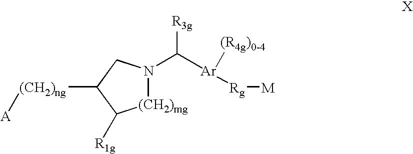

wherein
Ar is phenyl or naphthyl; each of m_{g} and n_{g} independently is 0 or 1; A is selected from COOH, PO₃H₂, PO₂H, SO₃H, PO(C₁₋₃alkyl)OH and 1H-tetrazol-5-yl; each of R_{1g} and R_{2g} independently is H, halogen, OH, COOH or C₁₋₄alkyl optionally substituted by halogen; R_{3g} is H or C₁₋₄alkyl optionally substituted by halogen or OH; each R_{4g} independently is halogen, or optionally halogen substituted C₁₋₄alkyl or C₁₋₃alkoxy; and each of R_{g} and M has one of the significances as indicated for B and C, respectively, in WO03/062252A1;
Compounds as disclosed in WO 03/062248A2, e.g. a compound of formula XI

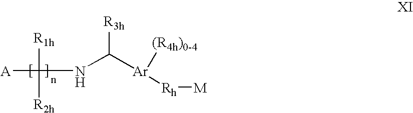

wherein Ar is phenyl or naphthyl; n is 2, 3 or 4; A is COOH, 1H-tetrazol-5-yl, PO₃H₂, PO₂H₂, —SO₃H or PO(R₅ₕ)OH wherein R₅ₕ is selected from C₁₋₄alkyl, hydroxyC₁₋₄alkyl, phenyl, —CO—C₁₋₃alkoxy and —CH(OH)-phenyl wherein said phenyl or phenyl moiety is optionally substituted;
each of R₁ₕ and R₂ₕ independently is H, halogen, OH, COOH, or optionally halogeno substituted C₁₋₆alkyl or phenyl; R₃ₕ is H or C₁₋₄alkyl optionally substituted by halogen and/OH; each R₄ₕ independently is halogeno, OH, COOH, C₁₋₄alkyl, S(O)_{0,1 or2}C₁₋₃alkyl, C₁₋₃alkoxy, C₃₋₆cycloalkoxy, aryl or aralkoxy, wherein the alkyl portions may optionally be substituted by 1-3 halogens; and each of Rₕ and M has one of the significances as indicated for B and C, respectively, in WO03/062248A2.

According to a further embodiment of the invention, a S1P receptor agonist or modulator for use in a combination of the invention may also be a selective S1P1 receptor, e.g. a compound which possesses a selectivity for the S1P1 receptor over the S1P3 receptor of at least 20 fold, e.g. 100, 500, 1000 or 2000 fold, as measured by the ratio of EC₅₀ for the S1P1 receptor to the EC₅₀ for the S1P3 receptor as evaluated in a ³⁵S-GTPγS binding assay, said compound having an EC₅₀ for binding to the S1P1 receptor of 100 nM or less as evaluated by the ³⁵S-GTPγS binding assay. Representative S1P1 receptor agonists or modulators are e.g. the compounds listed in WO 03/061567, the contents of which being incorporated herein by reference, for instance a compound of formula

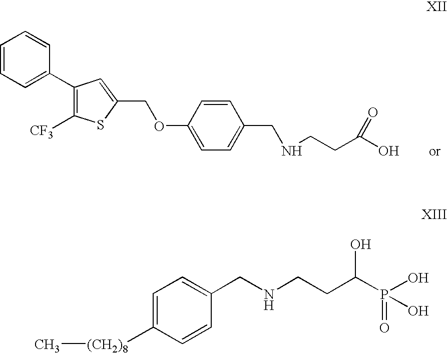

When the compounds of formulae I to XIII have one or more asymmetric centers in the molecule, the present invention is to be understood as embracing the various optical isomers, as well as racemates, diastereoisomers and mixtures thereof are embraced. Compounds of formula III or IVb, when the carbon atom bearing the amino group is asymmetric, have preferably the R-configuration at this carbon atom.

The compounds of formulae I to XIII may exist in free or salt form. Examples of pharmaceutically acceptable salts of the compounds of the formulae I to XIII include salts with inorganic acids, such as hydrochloride, hydrobromide and sulfate, salts with organic acids, such as acetate, fumarate, maleate, benzoate, citrate, malate, methanesulfonate and benzenesulfonate salts, or, when appropriate, salts with metals such as sodium, potassium, calcium and aluminium, salts with amines, such as triethylamine and salts with dibasic amino acids, such as lysine. The compounds and salts of the combination of the present invention encompass hydrate and solvate forms.

Acyl as indicated above may be a residue R_{y}—CO— wherein R_{y} is C₁₋₆alkyl, C₃₋₆cycloalkyl, phenyl or phenyl-C₁₋₄alkyl. Unless otherwise stated, alkyl, alkoxy, alkenyl or alkynyl may be straight or branched.

Aryl may be phenyl or naphthyl, preferably phenyl.

When in the compounds of formula I the carbon chain as R₁ is substituted, it is preferably substituted by halogen, nitro, amino, hydroxy or carboxy. When the carbon chain is interrupted by an optionally substituted phenylene, the carbon chain is preferably unsubstituted. When the phenylene moiety is substituted, it is preferably substituted by halogen, nitro, amino, methoxy, hydroxy or carboxy.

Preferred compounds of formula I are those wherein R₁ is C₁₃₋₂₀alkyl, optionally substituted by nitro, halogen, amino, hydroxy or carboxy, and, more preferably those wherein R₁ is phenylalkyl substituted by C₆₋₁₄-alkyl chain optionally substituted by halogen and the alkyl moiety is a C₁₋₆alkyl optionally substituted by hydroxy. More preferably, R₁ is phenyl-C₁₋₆alkyl substituted on the phenyl by a straight or branched, preferably straight, C₆₋₁₄alkyl chain. The C₆₋₁₄alkyl chain may be in ortho, meta or para, preferably in para.

Preferably each of R₂ to R₅ is H.

In the above formula of VII “heterocyclic group” represents a 5- to 7 membered heterocyclic group having 1 to 3 heteroatoms selected from S, O and N. Examples of such heterocyclic groups include the heteroaryl groups indicated above, and heterocyclic compounds corresponding to partially or completely hydrogenated heteroaryl groups, e.g. furyl, thienyl, pyrrolyl, azepinyl, pyrazolyl, imidazolyl, oxazolyl, isoxazolyl, thiazolyl, isothiazolyl, 1,2,3-oxadiazolyl, triazolyl, tetrazolyl, thiadiazolyl, pyranyl, pyridyl, pyridazinyl, pyrimidinyl, pyrazinyl, tetrahydropyranyl, morpholinyl, thiomorpholinyl, pyrrolidinyl, pyrrolyl, imidazolidinyl, pyrazolidinyl, piperidinyl, piperazinyl, oxazolidinyl, isoxazolidinyl, thiazolidinyl or pyrazolidinyl. Preferred heterocyclic groups are 5- or 6-membered heteroaryl groups and the most preferred heterocyclic group is a morpholinyl, thiomorpholinyl or piperidinyl group.

A preferred compound of formula I is 2-amino-2-tetradecyl-1,3-propanediol. A particularly preferred S1P receptor agonist of formula I is FTY720, i.e. 2-amino-2-[2-(4-octylphenyl)ethyl]propane-1,3-diol (referred to hereinafter as Compound A) in free form or in a pharmaceutically acceptable salt form, e.g. the hydrochloride, as shown:

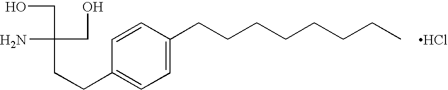

A preferred compound of formula II is the one wherein each of R′₂ to R′₅ is H and m is 4, i.e. 2-amino-2-{2-[4-(1-oxo-5-phenylpentyl)phenyl]ethyl}propane-1,3-diol, in free form or in pharmaceutically acceptable salt form (referred to hereinafter as Compound B), e.g. the hydrochloride.

A preferred compound of formula III is the one wherein W is CH₃, each of R″₁ to R″₃ is H, Z₂ is ethylene, X is heptyloxy and Y is H, i.e. 2-amino-4-(4-heptyloxyphenyl)-2-methyl-butanol, in free form or in pharmaceutically acceptable salt form (referred to hereinafter as Compound C), e.g. the hydrochloride. The R-enantiomer is particularly preferred.

A preferred compound of formula IVa is the FTY720-phosphate (R₂ₐ is H, R₃ₐ is OH, Xₐ is O, R₁ₐ and R_{1b} are OH). A preferred compound of formula IVb is the Compound C-phosphate (R₂ₐ is H, R_{3b} is OH, Xₐ is O, R₁ₐ and R_{1b} are OH, Yₐ is O and R₄ₐ is heptyl). A preferred compound of formula V is Compound B-phosphate.

A preferred compound of formula V is phosphoric acid mono-[(R)-2-amino-2-methyl-4-(4-pentyloxy-phenyl)-butyl]ester.

A preferred compound of formula VIII is (2R)-2-amino-4-[3-(4-cyclohexyloxybutyl)-benzo[b]thien-6-yl]-2-methylbutan-1-ol.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases.

The pharmaceutical activities as effected by administration of the combination according to the present invention can be demonstrated e.g. by using corresponding pharmacological models known in the pertinent art. The person skilled in the pertinent art is fully enabled to select a relevant animal test model to prove the hereinbefore and hereinafter indicated therapeutic indications and beneficial effects.

The dosage of the immunosuppressive or immunomodulator agent administered will also be generally dependent upon the health of the subject being treated, the extent of treatment desired, the nature and kind of concurrent therapy, if any, and the frequency of treatment and nature of the effect desired. In general, the dosage of the agent is generally in the range of from about 0.001 to about 50 mg/kg body weight of the subject per day, preferably from about 0.1 to about 10 mg/kg body weight of the subject per day, administered as a single or divided dose. However, some variability in the general dosage range may also be required depending upon the age, weight, and species of the patient, the intended route of administration, and the progress and degree of severity of the disease or condition being treated.

Daily dosages of the immunosuppressive or immunomodulator agent required in practicing the method of the present invention will vary depending upon, for example the mode of administration and the severity of the condition to be treated. An indicated daily dose is in the range of from about 0.1 to about 200 mg, e.g. from 0.1 to 100 mg of active agent for oral use, conveniently administered once or in divided dosages.

Preferred are combinations, such as combined preparations or pharmaceutical compositions, respectively, comprising a DPP-IV inhibitor preferably vildagliptin or a pharmaceutically accepted salt thereof and as second active agent selected from the group consisting of 2-amino-2-tetradecyl-1,3-propanediol, FTY720 i.e. 2-amino-2-[2-(4-octylphenyl)ethyl]propane-1,3-diol (referred to hereinafter as Compound A), the hydrochloride of FTY720 i.e. 2-amino-2-[2-(4-octylphenyl)ethyl]propane-1,3-diol, chlorhydrate (known under the INN name: Fingolimod), phosphoric acid mono-[(R)-2-amino-2-methyl-4-(4-pentyloxy-phenyl)-butyl]ester, (2R)-2-amino-4-[3-(4-cyclohexyloxybutyl)benzo[b]thien-6-yl]-2-methylbutan-1-ol, FTY720-phosphate, 2-amino-4-(4-heptyloxyphenyl)-2-methyl-butanol (referred to hereinafter as Compound C), the hydrochloride salt of 2-amino-4-(4-heptyloxyphenyl)-2-methyl-butanol the R-enantiomer of 2-amino-4-(4-heptyloxyphenyl)-2-methyl-butanol, Compound C-phosphate, 2-amino-2-{2-[4-(1-oxo-5-phenylpentyl)phenyl]ethyl}propane-1,3-diol (referred to hereinafter as Compound B), Compound B-hydrochloride, Compound B-phosphate, Rapamycin or Rapamycin derivatives, tacrolimus, Cyclosporins e.g. Cyclosporin “A”, Cyclosporin G, [O-(2-hydroxyethyl)-(D)Ser]⁸-Ciclosporin, and [3′-dehydroxy-3′-keto-MeBmt]¹-[Val]²-Ciclosporin, FK506, or in any case a pharmaceutically accepted salt thereof.

In a preferred embodiment, the immunomodulator is a S1P receptor agonist or modulator selected from a) is 2-amino-2-[2-(4-octylphenyl)ethyl]propane-1,3-diol, 2-amino-2-[4-(3-benzyloxyphenoxy)-2-chlorophenyl]propyl-1,3-propane-diol or 2-amino-2-[4-(benzyloxyphenylthio)-2-chlorophenyl]propyl-1,3-propane-diol, in free form or in a pharmaceutically acceptable salt or hydrate or crystal form.

The corresponding active ingredients or a pharmaceutically acceptable salt thereof may also be used in form of a solvate, such as a hydrate or including other solvents, used for crystallization.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases.

All of these marketed products may be utilized in as such for combination therapy according to the present invention.

The structure of the active agents identified by generic or tradenames may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

All the more surprising is the experimental finding that the combined administration of a DPP IV inhibitor or a salt thereof and at least one active ingredient selected from an immunosuppressive or immunomodulator agent or a salt thereof, results not only in a beneficial, especially a synergistic, therapeutic effect, but also in additional benefits resulting from the combined treatment and further surprising beneficial effects compared to a monotherapy applying only one of the pharmaceutically active compounds used in the combinations disclosed herein.

It can be shown by established test models and especially those test models described herein that the combination of the DPP-IV inhibitor with at least one active ingredient selected from an immunosuppressive or immunomodulator agent, results in a more effective prevention or preferably treatment of diseases specified in the following. In particular, it can be shown by established test models and especially those test models described herein that the combination of the present invention results in a more effective prevention or preferably treatment of diseases specified hereinafter.

If taken simultaneously, this results not only in a further enhanced beneficial, especially a synergistic, therapeutic effect, but also in additional benefits resulting from the simultaneous treatment such as a surprising prolongation of efficacy, a broader variety of therapeutic treatment and surprising beneficial effects on autoimmune diseases, e.g. insulitis, type I diabetes, LADA and the disorders associated with diabetes, or on graft rejection and conditions/disorders that might be treated by DPP-IV inhibition, in particular obesity, diabetes especially IGT and diseases and conditions associated with diabetes mellitus, IGT, obesity, Parkinson's disease, schizophrenia, Alzheimer's disease, and for a number of combinations as described herein.

The term “potentiation” shall mean an increase of a corresponding pharmacological activity or therapeutical effect, respectively. Potentiation of one component of the combination according to the present invention by co-administration of another component according to the present invention means that an effect is being achieved that is greater than that achieved with one component alone.

The term “synergistic” shall mean that the drugs, when taken together, produce a total joint effect that is greater than the sum of the effects of each drug when taken alone.

Moreover, for a human patient, especially for elderly people, it is more convenient and easier to remember to take two tablets at the same time, e.g. before a meal, than staggered in time, i.e. according to a more complicated treatment schedule. More preferably, both active ingredients are administered as a fixed combination, i.e. as a single tablet, in all cases described herein. Taking a single tablet is even easier to handle than taking two tablets at the same time. Furthermore, the packaging can be accomplished with less effort.

The person skilled in the pertinent art is fully enabled to select a relevant and standard animal test model to prove the hereinbefore and hereinafter indicated therapeutic indications and beneficial effects.

The pharmaceutical activities as effected by administration of the combination of the active agents used according to the present invention can be demonstrated e.g. by using corresponding pharmacological models known in the pertinent art.

The insulin secretion enhancing properties of the combination according to the present invention may be determined by following the methodology as disclosed, for example, in the publication of T. Ikenoue et al. Biol. Pharm. Bull. 29(4), 354-359 (1997).

The corresponding subject matter of these references is herewith incorporated by reference in this specification.

Accordingly, the combination according to the present invention may be used, e.g., for the prevention, delay of progression or treatment of diseases and disorders that may be inhibited by DPP IV inhibition and/or appetency disorders or nicotinic addiction.

Thus in a further aspect the present invention concerns the use of a combination comprising
i) a DPP IV inhibitor or a pharmaceutically acceptable salt thereof, and
ii) at least one active ingredient selected from an immunosuppressive or immunomodulator agent, or a pharmaceutically acceptable salt thereof
   for the manufacture of a medicament for the prevention, delay of progression or treatment of diseases and disorders that may be inhibited by DPP IV inhibition, for the prevention, delay of progression or treatment of autoimmune diseases, e.g. type 1 and the disorders associated therewith, or for the prevention, delay of progression or treatment of graft rejection. Conditions/disorders that might be treated by DPP-IV inhibition, are in particular obesity, diabetes especially type II diabetes, IGT and diseases and conditions associated with diabetes mellitus, Parkinson's disease, schizophrenia, Alzheimer's disease.

The invention furthermore relates to a method for the prevention, delay of progression or treatment of diseases and disorders that may be inhibited by DPP IV inhibition, for the prevention, delay of progression or treatment of autoimmune diseases, e.g. type 1 and the disorders associated therewith, or for the prevention, delay of progression or treatment of graft rejection, comprising administering to a warm-blooded animal, including man, in need thereof a jointly effective amount of a combination of a DPP IV inhibitor or a pharmaceutically acceptable salt thereof with at least one active ingredient selected from an immunosuppressive or immunomodulator agent, or a pharmaceutically acceptable salt thereof;

and at least one additional pharmaceutically acceptable carrier.

Conditions/disorders that might be treated by DPP-IV inhibition as mentioned herein, are in particular obesity, diabetes especially type II diabetes, IGT and diseases and conditions associated with diabetes mellitus, Parkinson's disease, schizophrenia, Alzheimer's disease.

The invention furthermore relates to a pharmaceutical composition for the prevention, delay of progression or treatment of diseases and disorders that may be inhibited by DPP IV inhibition, for the prevention, delay of progression or treatment of autoimmune diseases, e.g. type 1 and the disorders associated therewith, or for the prevention, delay of progression or treatment of graft rejection, comprising a combination of a DPP IV inhibitor or a pharmaceutically acceptable salt thereof with at least one active ingredient selected from an immunosuppressive or immunomodulator agent, or a pharmaceutically acceptable salt thereof;

and at least one additional pharmaceutically acceptable carrier.

Methods or uses as described above, wherein the disease or condition is selected from insulin resistance, impaired glucose metabolism, conditions of impaired glucose tolerance, conditions of impaired fasting plasma glucose, diabetes particularly type 2 diabetes mellitus, obesity, diabetic retinopathy, macular degeneration, cataracts, diabetic nephropathy, glomerulosclerosis, diabetic neuropathy, erectile dysfunction, premenstrual syndrome, coronary heart disease, hypertension, angina pectoris, myocardial infarction, stroke, vascular restenosis, skin and connective tissue disorders, foot ulcerations, ulcerative colitis, endothelial dysfunction, impaired vascular compliance, neurodegenerative disorders, cognitive disorders, memory and learning ability problems, autoimmune diseases, e.g. insulitis, type I diabetes, LADA, and the diseases or conditions associated with diabetes, or on graft rejection.

Methods or uses as described above, wherein the disease or condition is selected from impaired glucose metabolism, conditions of impaired glucose tolerance (IGT), conditions of impaired fasting plasma glucose, diabetes particularly type 2 diabetes mellitus, obesity, diabetic retinopathy, diabetic nephropathy, diabetic neuropathy, foot ulcerations, diseases or conditions associated with diabetes, Parkinson's disease, schizophrenia, Alzheimer's disease, dementia, senile dementia, mild cognitive impairment or Alzheimer type dementia, cognitive deficits associated with schizophrenia, impaired cognitive function associated with Alzheimer's disease, impaired cognitive function associated with Parkinson's disease, autoimmune diseases, e.g. insulitis, type I diabetes, LADA, and the diseases or conditions associated with diabetes, or on graft rejection.

Most preferably, the disease or condition is selected from obesity, diabetes, IGT, type 2 diabetes, Parkinson's disease, schizophrenia, Alzheimer's disease, insulitis, type 1 diabetes, LADA, diseases or conditions associated with diabetes and graft rejection.

In one further embodiment, the herein described methods, uses and compositions are used for the prevention of, delay of progression of, treatment of obesity, IGT, type 2 diabetes, insulitis, type 1 diabetes, LADA, graft rejection or diseases or conditions associated with diabetes.

In a preferred embodiment the herein described methods, uses and compositions are used for the prevention of, delay of progression of, or treatment of, graft rejection in bone marrow transplantation or pancreatic islets transplantation i.e. improve success rate in islet transplantation.

In further embodiment, the herein described methods, uses and compositions are used for the prevention of, delay of progression of, or treatment of, pancreatic islets graft rejection.

In a further aspect the present invention also covers a method for prolonging the time a patient with type 1 diabetes is in remission, said method comprising administering to a type 1 diabetes patient in remission an amount of a combination comprising a DPP-IV inhibitor and at least one immunosuppressive or immunomodulator agent as herein described, to prolong the time said patient is in remission, where the patient is preferably newly diagnosed with type 1 diabetes when the combination is first administered to the patient. The invention also covers the use of a combination comprising a DPP-IV inhibitor and at least one immunosuppressive or immunomodulator agent as herein described, or a salt thereof, for the manufacture of a medicament for prolonging the time a patient with type 1 diabetes is in remission, where the patient is preferably newly diagnosed with type 1 diabetes when the combination is first administered to the patient.

In further embodiment, the herein described methods, uses and compositions are used for prolonging the time a patient with type I diabetes is in remission or for the treatment of patients newly diagnosed with type 1 diabetes (see hereinafter).

Preferred combinations for the described uses or methods are described herein.

A “disease or condition which may be inhibited by a DPP-IV inhibitor” as defined in this application comprises, but is not limited to insulin resistance, impaired glucose metabolism, conditions of impaired glucose tolerance (IGT), conditions of impaired fasting plasma glucose, diabetes particularly type 2 diabetes mellitus, obesity, diabetic retinopathy, macular degeneration, cataracts, diabetic nephropathy, glomerulosclerosis, diabetic neuropathy, erectile dysfunction, premenstrual syndrome, coronary heart disease, hypertension, angina pectoris, myocardial infarction, stroke, vascular restenosis, skin and connective tissue disorders, foot ulcerations and ulcerative colitis, endothelial dysfunction and impaired vascular compliance, diseases or conditions associated with diabetes, neurodegenerative disorders, cognitive disorders and memory and learning ability problems. The neurodegenerative disorder is selected from Parkinson's disease, schizophrenia, dementia, senile dementia, mild cognitive impairment, Alzheimer related dementia, Huntington's chorea, tardive dyskinesia, hyperkinesias, mania, Morbus Parkinson, steel-Richard syndrome, Down's syndrome, myasthenia gravis, nerve and brain trauma, vascular amyloidosis, cerebral hemorrhage with amyloidosis, brain inflammation, Friedrich's ataxia, acute confusion disorders, acute confusion disorders in which apoptotic necrocytosis plays a part, amyotrophic lateral sclerosis, glaucoma, and Alzheimer's disease. The cognitive disorder is selected from cognitive deficits associated with schizophrenia, age-induced memory impairment, cognitive deficits associated with psychosis, cognitive impairment associated with diabetes, cognitive deficits associated with post-stroke, memory defects associated with hypoxia, cognitive and attention deficits associated with senile dementia, attention-deficit disorders, memory problems associated with mild cognitive impairment, impaired cognitive function associated with dementias, impaired cognitive function associated with Alzheimer's disease, impaired cognitive function associated with Parkinson's disease, impaired cognitive function associated with vascular dementia, cognitive problems associated with brain tumors, Pick's disease, cognitive deficits due to autism, cognitive deficits post electroconvulsive therapy, cognitive deficits associated with traumatic brain injury, amnesic disorders, deliriums, dementias.

Preferably, a “disease or condition which may be inhibited by a DPP-IV inhibitor” is selected from impaired glucose metabolism, conditions of impaired glucose tolerance, conditions of impaired fasting plasma glucose, diabetes particularly type 2 diabetes mellitus, obesity, diabetic retinopathy, diabetic nephropathy, diabetic neuropathy, foot ulcerations, diseases or conditions associated with diabetes, Parkinson's disease, schizophrenia, Alzheimer's disease, dementia, senile dementia, mild cognitive impairment or Alzheimer type dementia, cognitive deficits associated with schizophrenia, impaired cognitive function associated with Alzheimer's disease, impaired cognitive function associated with Parkinson's disease.

The term “curative” as used herein means efficacy in treating ongoing diseases, disorder or conditions.

The term “prophylactic” means the prevention of the onset or recurrence of diseases, disorders or conditions to be treated.

The term “delay of progression” as used herein means administration of the combination to patients being in a pre-stage or in an early phase of the disease to be treated, in which patients for example a pre-form of the corresponding disease is diagnosed or which patients are in a condition, e.g. during a medical treatment or a condition resulting from an accident, under which it is likely that a corresponding disease will develop.

The term “autoimmune diseases” include and is preferably selected from sarcoidosis, fibroid lung, idiopathic interstitial pneumonia, obstructive airways disease, including conditions such as asthma, intrinsic asthma, extrinsic asthma, dust asthma, particularly chronic or inveterate asthma (for example late asthma and airway hyperreponsiveness), bronchitis, including bronchial asthma, infantile asthma, allergic rheumatoid arthritis, systemic lupus erythematosus, nephrotic syndrome lupus, Hashimoto's thyroiditis, multiple sclerosis, myasthenia gravis, insulitis, type I diabetes mellitus and complications associated therewith, type II adult onset diabetes mellitus, latent autoimmune diabetes in adults (LADA), uveitis, nephrotic syndrome, steroid dependent and steroid-resistant nephrosis, palmoplantar pustulosis, allergic encephalomyelitis, glomerulonephritis, psoriasis, psoriatic arthritis, atopic eczema (atopic dermatitis), contact dermatitis and further eczematous dermatitises, seborrheic dermatitis, lichen planus, pemphigus, bullous pemphigoid, epidermolysis bullosa, urticaria, angioedemas, vasculitides, erythemas, cutaneous eosinophilias, acne, alopecia greata, eosinophilic fasciitis, atherosclerosis, conjunctivitis, keratoconjunctivitis, keratitis, vernal conjunctivitis, uveitis associated with Behcet's disease, herpetic keratitis, conical cornea, dystorphia epithelialis corneae, keratoleukoma, ocular pemphigus, Mooren's ulcer, scleritis, Graves' ophthalmopathy, severe intraocular inflammation, inflammation of mucosa or blood vessels such as leukotriene B4-mediated diseases, gastric ulcers, vascular damage caused by ischemic diseases and thrombosis, ischemic bowel disease, inflammatory bowel disease (e.g. Crohn's disease and ulcerative colitis), necrotizing enterocolitis, renal diseases including interstitial nephritis, Goodpasture's syndrome hemolytic uremic syndrome and diabetic nephropathy, nervous diseases selected from multiple myositis, Guillain-Barre syndrome, Meniere's disease and radiculopathy, collagen disease including scleroderma, Wegener's granuloma and Sjogren' syndrome, chronic autoimmune liver diseases including autoimmune hepatitis, primary biliary cirrhosis and sclerosing cholangitis), partial liver resection, acute liver necrosis (e.g. necrosis caused by toxins, viral hepatitis, shock or anoxia), B-virus hepatitis, non-A/non-B hepatitis and cirrhosis, fulminant hepatitis, pustular psoriasis, Behcet's disease, active chronic hepatitis, Evans syndrome, pollinosis, idiopathic hypoparathyroidism, Addison disease, autoimmune atrophic gastritis, lupoid hepatitis, tubulointerstitial nephritis, membranous nephritis, amyotrophic lateral sclerosis or rheumatic fever. It also covers prolonging the time a patient with type I diabetes is in remission or to treat patients newly diagnosed with type 1 diabetes.

By graft rejection is meant acute or chronic rejection of cells, tissue or solid organ allo- or xenografts of e.g. pancreatic islets, stem cells, bone marrow, skin, muscle, corneal tissue, neuronal tissue, heart, lung, combined heart-lung, kidney, liver, bowel, pancreas, trachea or oesophagus, or graft-versus-host diseases. Chronic rejection may also be named graft vessel diseases or graft vasculopathies.

In a preferred embodiment, graft rejection in bone marrow transplantation.

In an additional embodiment, the present invention concerns;

1. the use of a DPP IV inhibitor or a pharmaceutically acceptable salt thereof, for the manufacture of a medicament for the prevention, delay of progression or treatment of autoimmune diseases, e.g. insulitis, type I diabetes and the disorders associated therewith, or to improve pancreatic islets transplantation, preferably the autoimmune disease is latent autoimmune diabetes in adults (LADA).

2. a method for the prevention of, delay of progression of, or treatment of, autoimmune diseases, e.g. insulitis, type I diabetes and the disorders associated therewith, or to improve pancreatic islets transplantation, preferably the autoimmune disease is latent autoimmune diabetes in adults (LADA), comprising administering to a warm-blooded animal, including man, in need thereof an effective amount of a DPP IV inhibitor or a pharmaceutically acceptable salt thereof and at least one additional pharmaceutically acceptable carrier.

In the above described mono-therapy use of a DPP-4, preferably vildagliptin, or the corresponding method of treatment, the autoimmune disease is preferably selected from insulitis, LADA, type I diabetes, type I diabetes in a newly diagnosed type I diabetic patient.

Use of a DPP-4, preferably vildagliptin, or the corresponding method of treatment, as described above, to improve pancreatic islets transplantation or to treat a patient having been subject to a pancreatic islets transplantation.

By the term “improve pancreatic islets transplantation”, the applicant means reducing any functional or physiological problems of the pancreatic islets (transplanted or the patients own islets) following to a pancreatic islets transplantation. It covers in particular the treatment of a patient having been subject to a pancreatic islets transplantation.

In a further aspect the present invention also covers a method for prolonging the time a patient with type 1 diabetes is in remission, said method comprising administering to a type I diabetes patient in remission an amount of a DPP-4 inhibitor especially viladgliptin, or a salt thereof, to prolong the time said patient is in remission, preferably where the patient is newly diagnosed with type 1 diabetes when the DPP-4 inhibitor is first administered to the patient. The invention also covers the use of a DPP-4 inhibitor especially viladgliptin, or a salt thereof, for the manufacture of a medicament for prolonging the time a patient with type 1 diabetes is in remission, where the patient is preferably newly diagnosed with type 1 diabetes when the DPP-4 inhibitor is first administered to the patient.

In one embodiment of the aforementioned use or method for prolonging the time a patient with type 1 diabetes is in remission, the patient is further administered an autoimmune agent.

By “newly diagnosed with type 1 diabetes” as used in the present application is meant that the patient has been diagnosed with type 1 diabetes within the last 12 months, preferably within the last 6 months, more preferably within the last 3 months, even more I preferably within the last 2 months, and most preferably within the last month.

Of course, one skilled in the art would recognize that type 1 diabetes can be diagnosed by one or more of the following tests, including but not limited to, a urinalysis showing the presence of glucose and ketone bodies in the urine, a fasting blood glucose of 126 mg/dl or higher, a random glucose of greater than 200 mg/dl, an HbA,C of greater than 6% (where the % is a % of total hemoglobin), a serum insulin test where the fasting insulin is greater than 20 mcU/ml, or a C-peptide test of greater than 100 μmol/l.

In one embodiment of the methods or uses of the invention, the patient is newly diagnosed with type 1 diabetes before 18 years of age. In another embodiment of the methods of the invention, the patient is newly diagnosed with type 1 diabetes before 16 years of age.

In a further embodiment, the patient is newly diagnosed with type 1 diabetes while the patient is prepubescent. In yet another embodiment, is newly diagnosed with type 1 diabetes before 12 years of age. In a further embodiment, the patient is newly diagnosed with type 1 diabetes before 6 years of age.

In a further embodiment, the patient to be treated is in remission where remission may be defined in a number of ways. For example, remission may be defined as an insulin requirement of 0.5 U/kg/24 h, or as an insulin requirement of 0.5 U/kg/24 h in combination with HbAC below 7.5%, or as a basal C-peptide level of >100 μmol/l. In a preferred embodiment, remission is defined by the formula: HbA,C+(4×the daily insulin dose (U/Kg/24 h) 9%. Where the patient to be treated with the DPP-4 inhibitor or a combination as described herein is in remission, it is believed that treatment of the patient with the DPP-4 inhibitor or the herein described combination will prolong the period said patient is in remission (“the remission period”) relative to treatment in the absence of the DPP-4 inhibitor or the herein described combination.

Accordingly, the present invention also relates to a use or method for prolonging the time a patient with type I diabetes is in remission, said method comprising administering to a type 1 diabetes patient in remission an amount of a DPP-4 inhibitor or the herein described combination, effective to prolong the time said patient is in remission, where said remission is measured by one of the formulas described above.

The term “combined pharmaceutical preparation” as that term is used herein means that the active ingredients, e.g. 2-amino-2-[2-(4-octylphenyl)ethyl]propane-1,3-diol, 2-amino-2-[4-(3-benzyloxyphenoxy)-2-chlorophenyl]propyl-1,3-propane-diol or 2-amino-2-[4-(benzyloxyphenylthio)-2-chlorophenyl]propyl-1,3-propane-diol and a DPP-IV inhibitor preferably vildagliptin, or in each case, a pharmaceutically acceptable salt thereof, are both administered to a patient as separate entities either simultaneously, concurrently or sequentially with no specific time limits, wherein such administration provides therapeutically effective levels of the two compounds in the body, preferably at the same time. As an example, a non-fixed combination would be two capsules each containing one active ingredient where the purpose is to have the patient achieve treatment with both active ingredients together in the body.

The term “treat” or “treatment” encompasses the complete range of therapeutically positive effects associated with pharmaceutical medication including reduction of, alleviation of and relief from the symptoms or illness which affect the organism.

Preferably, the jointly therapeutically effective amounts of the active agents according to the combination of the present invention can be administered simultaneously or sequentially in any order, e.g. separately (combined pharmaceutical preparation) or in a fixed combination.

Under certain circumstances, drugs with different mechanisms of action may be combined. However, just considering any combination of drugs having different modes of action but acting in the similar field does not necessarily lead to combinations with advantageous effects.

All the more surprising is the experimental finding that the combined administration of a DPP-IV inhibitor according to the present invention, or, in each case, a pharmaceutically acceptable form thereof, results not only in a beneficial, especially a potentiating or a synergistic, therapeutic effect. Independent thereof, additional benefits resulting from combined treatment can be achieved such as a surprising prolongation of efficacy, a broader variety of therapeutic treatment and surprising beneficial effects on diseases and conditions associated with diabetes (e.g. less appetite, less gain of weight or less cardiovascular side effects, less beta cells necrosis/apoptosis, improved beta cells neogenesis).

The diseases, disorders or conditions related (associated) to diabetes type 1 or type 2, includes but are not limited to diabetic nephropathy, diabetic retinopathy and diabetic neuropathy, macular degeneration, coronary heart disease, myocardial infarction, diabetic cardiomyopathy, myocardial cell death, coronary artery diseases, peripheral arterial disease, stroke, limb ischemia, vascular restenosis, foot ulcerations, endothelial dysfunction and/or atherosclerosis.

Further benefits are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

For example, it has turned out that the combination according to the present invention provides benefit especially in the treatment of diabetic patients, e.g. reducing the risk of negative cardiovascular events, reducing risk of side effects, controlling increase of weight (in diabetic patients) or in patients suffering from an altered gastrointestinal motility, sensitivity and/or secretion disorder(s).

In view of reduced dose of the DPP-IV inhibitor or immunosuppressive or immunomodulator agent, used according to the present invention, there is a considerable safety profile of the combination making it suitable for first line therapy.

The pharmaceutical composition according to the present invention as described herein before and hereinafter may be used for simultaneous use or sequential use in any order, for separate use or as a fixed combination.

Method or use as described above, wherein the DPP-IV inhibitor and the immunosuppressive or immunomodulator agent are administered in the form of a combination of the present invention such as a fixed combination or combined preparation or kit of part.

“kit-of-parts”, combination, method or use as described herein, wherein the DPP-IV inhibitor is vildagliptin or and wherein the immunosuppressive or immunomodulator agent is preferably selected from the group consisting of 2-amino-2-tetradecyl-1,3-propanediol, FTY720, i.e. 2-amino-2-[2-(4-octylphenyl)ethyl]propane-1,3-diol (referred to hereinafter as Compound A), the hydrochloride of FTY720, phosphoric acid mono-[(R)-2-amino-2-methyl-4-(4-pentyloxy-phenyl)-butyl]ester, (2R)-2-amino-4-[3-(4-cyclohexyloxybutyl)-benzo[b]thien-6-yl]-2-methylbutan-1-ol, FTY720-phosphate, 2-amino-4-(4-heptyloxyphenyl)-2-methyl-butanol (referred to hereinafter as Compound C), the hydrochloride salt of 2-amino-4-(4-heptyloxyphenyl)-2-methyl-butanol the R-enantiomer of 2-amino-4-(4-heptyloxyphenyl)-2-methyl-butanol, Compound C-phosphate, 2-amino-2-{2-[4-(1-oxo-5-phenylpentyl)phenyl]ethyl}propane-1,3-diol (referred to hereinafter as Compound B), Compound B-hydrochloride, Compound B-phosphate, Rapamycin or Rapamycin derivatives, tacrolimus, Cyclosporins e.g. Cyclosporin “A”, Cyclosporin G, [O-(2-hydroxyethyl)-(D)Ser]⁸-Ciclosporin, and [3′-dehydroxy-3′-keto-MeBmt]¹-[Val]²-Ciclosporin, FK506, or in each case, a pharmaceutically acceptable salt thereof.

“kit-of-parts”, combination, method or use as described above, wherein the DPP-IV inhibitor is vildagliptin and wherein the immunosuppressive or immunomodulator agent is 2-amino-2-[2-(4-octylphenyl)ethyl]propane-1,3-diol, 2-amino-2-[4-(3-benzyloxyphenoxy)-2-chlorophenyl]propyl-1,3-propane-diol or 2-amino-2-[4-(benzyloxyphenylthio)-2-chlorophenyl]propyl-1,3-propane-diol, or in each case, a pharmaceutically acceptable salt thereof.

According the invention, when the DPP-IV inhibitors, and the immunosuppressive or immunomodulator agent are administered together, such administration can be sequential in time or simultaneous with, the simultaneous method being generally preferred. For sequential administration, the DPP-IV inhibitor, and the immunosuppressive or immunomodulator agent can be administered in any order. It is generally preferred that such administration be oral. It is especially preferred that the administration be oral and simultaneous. However, if the subject being treated is unable to swallow, or oral absorption is otherwise impaired or undesirable, parenteral or transdermal administration will be appropriate. When the DPP-IV inhibitor, and the immunosuppressive or immunomodulator agent are administered sequentially, the administration of each can be by the same method or by different methods.

A further aspect of the present invention is a kit for the prevention of, delay of progression of, treatment of a disease or condition according to the present invention comprising

(a) an amount of a DPP IV inhibitor or a pharmaceutically acceptable salt thereof in a first unit dosage form;
(b) an amount of at least one active ingredient selected from an immunosuppressive or immunomodulator agent or, in each case, where appropriate, a pharmaceutically acceptable salt thereof in a second etc. unit dosage form; and
(c) a container for containing said first, second etc. unit forms.

In a variation thereof, the present invention likewise relates to a “kit-of-parts”, for example, in the sense that the components to be combined according to the present invention can be dosed independently or by use of different fixed combinations with distinguished amounts of the components, i.e. simultaneously or at different time points. The parts of the kit of parts can then e.g. be administered simultaneously or chronologically staggered, that is at different time points and with equal or different time intervals for any part of the kit of parts. Preferably, the time intervals are chosen such that the effect on the treated disease or condition in the combined use of the parts is larger than the effect that would be obtained by use of only any one of the components.

The present invention thus also relates to a kit of parts comprising

(a) an amount of a DPP IV inhibitor or a pharmaceutically acceptable salt thereof in a first unit dosage form;
(b) an amount of at least one active ingredient selected from an immunosuppressive or immunomodulator agent or, in each case, where appropriate, a pharmaceutically acceptable salt thereof,
in the form of two or three or more separate units of the components (a) to (b), especially for the prevention of, delay of progression of, treatment of a disease or condition according to the present invention.

The invention furthermore relates to a commercial package comprising the combination according to the present invention together with instructions for simultaneous, separate or sequential use.

In a preferred embodiment, the (commercial) product is a commercial package comprising as active ingredients the combination according to the present invention (in the form of two or three or more separate units of the components (a) or (b)), together with instructions for its simultaneous, separate or sequential use, or any combination thereof, in the delay of progression or treatment of the diseases as mentioned herein.

All the preferences mentioned herein apply to the combination, composition, use, method of treatment, “kit of parts” and commercial package of the invention.

These pharmaceutical preparations are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compound. Pharmaceutical preparations for enteral or parenteral, and also for ocular, administration are, for example, in unit dose forms, such as coated tablets, tablets, capsules or suppositories and also ampoules. These are prepared in a manner that is known per se, for example using conventional mixing, granulation, coating, solubilizing or lyophilizing processes. Thus, pharmaceutical preparations for oral use can be obtained by combining the active compound(s) with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

Preferred dosages for the active ingredients of the pharmaceutical combination according to the present invention are therapeutically effective dosages, especially those which are commercially available.

Normally, in the case of oral administration, an approximate daily dose of from about 1 mg to about 360 mg is to be estimated e.g. for a patient of approximately 75 kg in weight.

The dosage of the active compound can depend on a variety of factors, such as mode of administration, homeothermic species, age and/or individual condition.

The pharmaceutical preparation will be supplied in the form of suitable dosage unit form, for example, a capsule or tablet, and comprising an amount, being together with the further component(s) jointly effective, e.g. 100 mg or 50 mg of vildagliptin.

The pharmaceutical composition according to the present invention as described hereinbefore may be used for simultaneous use or sequential use in any order, for separate use or as a fixed combination.

Thus according to a further embodiment, a DPP-IV inhibitor, is administered with at least one active ingredient selected from an immunosuppressive or immunomodulator agent, preferably in the form of a fixed pharmaceutical composition comprising a pharmaceutically acceptable carrier, vehicle or diluent. Accordingly, a DPP-IV inhibitor of this invention, can be administered with at least one active ingredient selected from an immunosuppressive or immunomodulator agent as a fixed combination, in any conventional oral, parenteral or transdermal dosage form.

The doses of DPP-IV inhibitor of formula (I) to be administered to warm-blooded animals, for example human beings, of, for example, approximately 70 kg body weight, especially the doses effective in the inhibition of the DPP-IV enzyme, are from approximately 3 mg to approximately 3 g, preferably from approximately 10 mg to approximately 1 g, for example approximately from 20 mg to 200 mg, per person per day, divided preferably into 1 to 4 single doses which may, for example, be of the same size. Usually, children receive about half of the adult dose. The dose necessary for each individual can be monitored, for example by measuring the serum concentration of the active ingredient, and adjusted to an optimum level. Single doses comprise, for example, 10, 40 or 100 mg per adult patient.

The dosage of vildagliptin is preferably between 10 and 150 mg daily, most preferably between 25 and 150 mg, 25 and 100 mg or 25 and 50 mg or 50-100 mg daily. Preferred examples of daily oral dosage are 25, 30, 35, 45, 50, 55, 60, 80, 100 or 150 mg. The application of the active ingredient may occur up to three times a day, preferably one or two times a day.

The S1P receptor agonist or modulator, e.g. a compound of formulae I to XIII, e.g. Compound A or B, may be administered by any conventional route, in particular enterally, e.g. orally, e.g. in the form of tablets, capsules, drink solutions or parenterally, e.g. in the form of injectable solutions or suspensions. Suitable unit dosage forms for oral administration comprise from ca. 0.01 to 50 mg active ingredient, usually 0.1 to 30 mg, e.g. Compound A or B, together with one or more pharmaceutically acceptable diluents or carriers therefore. A preferred combination is the combination of FTY720 and vildagliptin or in any case a salt thereof e.g. FTY720 hydrochloride.

The preferred herein mentioned immunosuppressive or immunomodulator agent will be supplied in the form of suitable dosage unit form, for example, a capsule or tablet, and comprising a therapeutically effective amount, e.g. from about 0.1 to about 100 mg, as already described herein and in the prior art. The application of the active ingredient may occur up to three times a day, preferably one or two times a day. The same preferred dosage are selected for the fixed combinations.

Daily 2-amino-2-[2-(4-octylphenyl)ethyl]propane-1,3-diol, 2-amino-2-[4-(3-benzyloxyphenoxy)-2-chlorophenyl]propyl-1,3-propane-diol or 2-amino-2-[4-(benzyloxyphenylthio)-2-chlorophenyl]propyl-1,3-propane-diol dosages required in practicing the method of the present invention will vary depending upon, for example the mode of administration and the severity of the condition to be treated. An indicated daily dose is in the range of from about 0.1 to about 100 mg, e.g. from 1 to 10 mg of active agent for oral use, conveniently administered once or in divided dosages. Preferably a daily dosage from about 0.5 to about 6 mg.

Corresponding doses may be taken, for example, in the morning, at mid-day or in the evening.

In a preferred aspect, the invention concerns a “kit-of-parts”, combination, use or a method as described herein, comprising or wherein the daily administration is;
i) between 25 and 150 mg or between 50 and 100 mg of vildagliptin, and
ii) between 0.5 to 10 mg, or between 0.5 and 6 mg of a compound selected from 2-amino-2-[2-(4-octylphenyl)ethyl]propane-1,3-diol, 2-amino-2-[4-(3-benzyloxyphenoxy)-2-chlorophenyl]propyl-1,3-propane-diol and 2-amino-2-[4-(benzyloxyphenylthio)-2-chlorophenyl]propyl-1,3-propane-diol,
   or in any case, a pharmaceutically acceptable salt thereof.

In a preferred aspect, the invention concerns a “kit-of-parts”, combination or use or a method as described herein, comprising or wherein the daily administration is;
i) 50 or 100 mg of vildagliptin, and
ii) 2.5 or 5 mg of a compound selected from 2-amino-2-[2-(4-octylphenyl)ethyl]propane-1,3-diol, 2-amino-2-[4-(3-benzyloxyphenoxy)-2-chlorophenyl]propyl-1,3-propane-diol and 2-amino-2-[4-(benzyloxyphenylthio)-2-chlorophenyl]propyl-1,3-propane-diol,
   or in any case, a pharmaceutically acceptable salt thereof.

Preferably amino-2-[2-(4-octylphenyl)ethyl]propane-1,3-diol, chlorhydrate, is administered in an amount between 2.5 mg and 5 mg daily.

Preferably, in case of free combinations, preferred are those dosages for launched products that have been approved and that have been marketed.

Especially preferred are low dose combinations.

To further illustrate the invention, but not by way of limitation, the following examples are provided.

Although the present invention has been described in considerable detail with reference to certain preferred versions thereof, other versions are possible without departing from the spirit and scope of the preferred versions contained herein. All references and patents (U.S. and others) referred to herein are hereby incorporated by reference in their entirety as if set forth herein in full.

Utility of the present can be demonstrated by following the examples of US20030180345.

Utility of the compounds combination of the invention in a method as hereinabove specified, may be demonstrated in animal test methods as well as in clinic, for example in accordance with the methods hereinafter described.

### A1. Islet Graft

Islets from BALB/C(H-2^{d}) mice are transplanted beneath the renal capsule of STZ-induced diabetic CBA (H-2^{k}) mice. The recipients are treated orally with a combination according to the invention for 50 days after islet transplantation, each component being preferably administered at a daily dose of 0.1 to 40 mg/kg. Functional status of the islet graft is monitored by measuring blood glucose daily. Normal glycemia can be maintained for more days in the treated animals compared to untreated animals, e.g. when animals are treated with 1 or 3 mg/kg/d of Compound A hydrochloride and 0.5 to 75 mg/kg/d vildagliptin.

### B. Combined Treatment

Suitable clinical studies are, for example, open label, dose escalation studies in patients with psoriasis or multiple sclerosis. Such studies prove in particular the synergism of the active ingredients of the combination of the invention. The beneficial effects on psoriasis or multiple sclerosis can be determined directly through the results of these studies which are known as such to a person skilled in the art. Such studies are, in particular, suitable to compare the effects of a monotherapy using the active ingredients and a combination of the invention. Preferably, the dose of agent (a) is escalated until the Maximum Tolerated Dosage is reached, and agent (b) is administered with a fixed dose. Alternatively, the agent (a) is administered in a fixed dose and the dose of agent (b) is escalated. Each patient receives doses of the agent (a) either daily or intermittent. The efficacy of the treatment can be determined in such studies, e.g., after 12, 18 or 24 weeks by evaluation of symptom scores every 6 weeks.

Alternatively, a placebo-controlled, double blind study can be used in order to prove the benefits of the combination of the invention mentioned herein, e.g. in transplantation of an organ, tissue or cells, e.g. Langerhans islet cells.

### C. Beta Cell Neogenesis and Islet Apoptosis—Treatment and Prevention of Autoimmune Diseases.

This study describes who vildagliptin and an immunomodulator can lead to an increase of beta cell mass and decrease of islet apoptosis. Pancreatic beta cell growth are evaluated using neonatal rats, a model for rapid beta cell turnover and growth. Neonatal rats (n=5-8/group) are orally dosed once-a-day with vildagliptin (60 mg/kg/day), 1 or 3 mg/kg/d of Compound A hydrochloride and with the combination 1 or 3 mg/kg/d of Compound A hydrochloride and vildagliptin (60 mg/kg/day) or vehicle (controls) from days 1 through 21. Pancreatic immunohistochemistry and morphometric analysis are performed on days 7, 21 and 28. At day 7 the number of BrdU-positive islet cells and the decrease of number of Apoptag-positive cells in the insulin-staining islet is analyzed. Following 21 days of dosing beta cell mass of treated rats and pancreatic insulin content is assessed. This study can support the beneficial effect of the claimed combinations on islet cells.

### Animals and Procedures

Timed-pregnant Wistar rats (Charles River) are individually housed from gestational day 14 with ad libitum access to standard rodent chow and water in a temperature and humidity-controlled environment with a 12-h light cycle.

Following delivery (˜gestational day 21=Day 0 of study) all pups remained undisturbed for 48 h.

Thirty-six pups (male and female, gender undetermined) are assigned to receive vehicle, and 35 pups are assigned to receive vildagliptin (60 mg/kg, po) 1 or 3 mg/kg of Compound A hydrochloride and with the combination 1 or 3 mg/kg of Compound A hydrochloride and vildagliptin (60 mg/kg), once daily, starting on Day 2.

One group of pups (n=12/treatment group) is euthanized on Day 7, after 5 days of treatment. A second cohort (n=12/treatment group) continue treatment until Day 21, and is euthanized after 19 days of treatment. The remaining animals (having received treatment from Days 2 to 20) are weaned, their gender determined, and housed 2 rats/cage on Day 21, and receive no further treatment. These animals are euthanized on Day 33, after 12 days of “wash-out”.

On the day of sacrifice, all animals are injected with 5′-bromo-2′-deoxyuridine (BrdU; 100 mg/kg, ip). One hour later, blood samples are obtained by cardiac puncture and animals are euthanized with CO₂ and pancreatic tissue are harvested.

Pancreata from half of each treatment group are weighed and fixed in 10% neutral buffered formalin for subsequent immunocytochemistry (IHC)-morphometric analysis.

Pancreas from half of each treatment group are weighed and frozen in liquid nitrogen for subsequent determination of pancreatic insulin content.

D. Treatment of Children with Newly Diagnosed Type 1 Diabetes and Prolonging the Time a Patient with Type 1 Diabetes is in Remission.

Pharmacological intervention during this period (remission) with vildadliptin or one of the herein described combinations is expected to boost/reinforce the survival of residual beta-cell mass, thereby resulting in prolongation of the remission period.

Study Design A total number of 100 children and adolescents (age below 16 yrs) with newly diagnosed type 1 diabetes are enrolled to participate in the study. Clinical information on sex, age, pubertal status, duration of symptoms, DKA at presentation, and insulin regimen will be recorded. Baseline C-peptide and glucose are determined at diagnosis. A stimulated C peptide test is carried out in each subject at 1, 6 and 12 months after diagnosis. HbA,C and serum for immunology (ICA, GAD, IA2, IM) are analysed with regular intervals in this period. At study entry blood samples are drawn for DNA isolation and HLA typing from each individual.

50 children are treated with insulin as usual during a period of 1 year and 3 groups of 50 children are randomized to treatment with vildagliptin (50 mg), compound A (1.5 mg) or the combination vildagliptin and compound A, in combination with insulin.

Statistics with power considerations The statistical evaluation is based on a repeated measurements model for data on stimulated C-peptide dose-adjusted HbA,C respectively. The analyses do correct for the baseline value. The power has been calculated in a slighter simpler frame, using only the 12-month value as response. This is the final and thus most informative value. It has been Calculated how large differences can be obtained with a study size of 200 patients (50 in each group). Values of the variation within and between patients are taken as found in the Hvidere remission in former studies.

For stimulated C-peptide (logarithmic scale), the variation within patients is 0.39 and between patients 0.59. This implies that the variation on the 12-month value is an SD of 0.62, when the baseline is accounted for. Using a significance level of 0.05 and a power of 0.9, a minimum difference of 0.51 implies that it is necessary to have 50 patients in each group.

This difference corresponds to a factor of 1.67 between the two treatment groups, in the stimulated C-peptide after 12 months. This evaluation does not account for dropout.

For dose-adjusted HbA1c (HbA1c %+4×daily dose/kg), the variation within patients is 1.74 and between patients 1.92. This implies that the variation on the 12-month value is an SD of 1.63, when the baseline is accounted for. Using a significance level of 0.05 and a power of 0.9, a minimum difference of 1.06 implies that it is necessary to have 50 patients in each group. This evaluation does not account for dropout.

Treatment with vildagliptin, the compound A or the combination comprising vildagliptin and the compound A can prolong the remission phase in children with newly diagnosed type I diabetes by stimulating the preservation of the patients' residual beta-cell function as measured by a surrogate marker for beta cell function, C peptide levels.

### E: Effect of Vildagliptin or the Combination “Vildagliptin+Compound A” on Insulitis.

To investigate the effect of Vildagliptin or the combination “Vildagliptin+compound A” on insulitis or type I diabetes mellitus, mice (NOD) with a genetic predisposition to develop IDDM (insulin dependent diabetes mellitus) are treated with Vildagliptin or the combination “Vildagliptin+compound A”.

The animals are dosed intraperitoneally from 4 weeks of age, 3 times a week for four weeks. Insulitis is assessed at 14 weeks and scored in a table.

The mice are scored according to the method of Beales et al. (European Journal of Pharmacology 357 (1998) 221-225. 3 or more (DMSO and PBS) represents severe grade insulitis and 1-3 represents little infiltration (a score of 1 would represent low grade peri-insulitis).

Vildagliptin and the combination of “Vildagliptin+compound A” can show unexpected good results in reducing insulitis.

F. Effect of Vildagliptin and the combination of “Vildagliptin+compound A” on incidence of Insulitis Diabetes-prone non-obese diabetic (NOD) mice in which females develop a 90% rate of autoimmune diabetes by the age of 25 weeks is studied in order to determine the effect of Vildagliptin and the combination of “Vildagliptin+compound A” on insulitis onset and development. Insulitis initiates at 3-5 weeks of age in NOD mice, as leukocytes begin to infiltrate around ducts and venules in both female and male mice. These infiltrates progress toward the islets, which become surrounded by concentric layers of pen-insular lymphocytes (non-destructive peri-insulitis). Destructive intra-islet insulitis then occurs, leading to extensive P cell destruction. All NOD mice display peri-insulitis, whereas intra-insulitis and overt Type 1 diabetes is restricted to about 70-80% of females and about 10-15% of males in the NOD mouse colony used in this instance.

Insulitic infiltrates consist mainly of CD4′ and CD8′ T cells, but include some macrophages, B cells and natural killer (NK) cells.

The NOD mouse model of diabetes is a well established model directly comparable to human Type I diabetes. The NOD mouse spontaneously develops a disease closely resembling Type 1 diabetes in histology and range of autoimmune responses. Ultimately, the NOD mouse exhibits a loss of cells in the pancreatic islets.

Pregnant NOD mice are maintained on a control diet either with or without Vildagliptin or the combination of “Vildagliptin+compound A” supplementation in the drinking water throughout pregnancy and lactation. Supplementation of Vildagliptin or the combination of “Vildagliptin+compound A” is stopped after weaning. At 12 weeks of age the animals are killed and examined for histological evidence of insulitis within the pancreatic islets. Mice which are examined and found to have evidence of insulitis, are then further scored as peri-islet (slight), less than 50% area of islet (medium) or more than 50% islet area (heavy), as indicative of the stage and/or severity of insulitis.

Vildagliptin and the combination of “Vildagliptin+compound A” can show unexpected good results in reducing insulitis.

1. Combinations comprising
i) a DPP IV inhibitor or a pharmaceutically acceptable salt thereof, and
ii) at least one active ingredient selected from an immunosuppressive or an immunomodulator agent, or a pharmaceutically acceptable salt thereof.

2. Combination according to claim 1 comprising
i) a DPP IV inhibitor or a pharmaceutically acceptable salt thereof, and
ii) at least one active ingredient selected from an immunosuppressive or an immunomodulator agent, or a pharmaceutically acceptable salt thereof,
and at least one additional pharmaceutically acceptable carrier.

3. Combination according to claim 1, in the form of a combined preparation or a fixed combination.

4. (canceled)

5. A method for the prevention, delay of progression or treatment of diseases and disorders that may be inhibited by DPP IV inhibition, for the prevention, delay of progression or treatment of autoimmune diseases, and the disorders associated therewith, or for the prevention, delay of progression or treatment of graft rejection, comprising administering to a warm-blooded animal, including man, in need thereof a jointly effective amount of a combination of a DPP IV inhibitor or a pharmaceutically acceptable salt thereof with at least one active ingredient selected from an immunosuppressive or immunomodulator agent, or a pharmaceutically acceptable salt thereof;
and at least one additional pharmaceutically acceptable carrier.

6. Method according to claim 5, wherein the disease or condition is selected from impaired glucose metabolism, conditions of impaired glucose tolerance, conditions of impaired fasting plasma glucose, diabetes particularly type 2 diabetes mellitus, obesity, diabetic retinopathy, diabetic nephropathy, diabetic neuropathy, foot ulcerations, diseases or conditions associated with diabetes, Parkinson's disease, schizophrenia, Alzheimer's disease, dementia, senile dementia, mild cognitive impairment or Alzheimer type dementia, cognitive deficits associated with schizophrenia, impaired cognitive function associated with Alzheimer's disease, impaired cognitive function associated with Parkinson's disease, appetency disorders or substance abuse disorders, or for body fat reduction.

7. Method according to claim 6, wherein the disease or condition is selected from obesity, IGT, type 2 diabetes, insulitis, type 1 diabetes, LADA, graft rejection or diseases or conditions associated with diabetes.

8. (canceled)

9. A method for prolonging the time a patient with type 1 diabetes is in remission, said method comprising administering to a type 1 diabetes patient in remission an amount of a combination comprising a DPP-IV inhibitor and at least one immunosuppressive or immunomodulator agent as herein described, to prolong the time said patient is in remission.

10. (canceled)

11. Method of claim 9, wherein the patient is newly diagnosed with type 1 diabetes when the combination is first administered to the patient.

12. (canceled)

13. A method for the prevention of, delay of progression of, or treatment of, autoimmune diseases, type I diabetes and the disorders associated therewith, or to improve pancreatic islets transplantation, comprising administering to a warm-blooded animal, including man, in need thereof an effective amount of a DPP IV inhibitor or a pharmaceutically acceptable salt thereof and at least one additional pharmaceutically acceptable carrier.

14. Method according to claim 13, wherein the autoimmune disease is latent autoimmune diabetes in adults (LADA).

15. Method according to claim 13, to improve pancreatic islets transplantation or to treat a patient having been subject to a pancreatic islets transplantation.

16. A method for prolonging the time a patient with type 1 diabetes is in remission, said method comprising administering to a type 1 diabetes patient in remission an amount of a DPP-IV inhibitor or a pharmaceutically acceptable salt thereof, to prolong the time said patient is in remission.

17. (canceled)

18. Method of claim 16, wherein the patient is newly diagnosed with type 1 diabetes when the DPP-IV inhibitor or a pharmaceutically acceptable salt thereof, is first administered to the patient.

19. Combination according to claim 1, wherein the DPP-IV inhibitor is selected from (S)-1-{2-[5-cyanopyridin-2-yl)amino]ethyl-aminoacetyl)-2-cyano-pyrrolidine, vildagliptin, MK-0431 (Sitagliptin), GSK23A, saxagliptin, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide, or in each case, a pharmaceutically acceptable salt thereof.

20. Combination according to claim 19, wherein the DPP-IV inhibitor is vildagliptin or a pharmaceutically acceptable salt thereof.

21. Combination according to claim 1, wherein the immunosuppressive or immunomodulator agent is selected from the group consisting of Mycophenolic acid or a salt or ester thereof, mycophenolate sodium, mycophenolate mofetil, 2-amino-2-tetradecyl-1,3-propanediol; 2-amino-2-[2-(4-octylphenyl)ethyl]propane-1,3-diol (FTY720); the hydrochloride of 2-amino-2-[2-(4-octylphenyl)ethyl]propane-1,3-diol; phosphoric acid mono-[(R)-2-amino-2-methyl-4-(4-pentyloxy-phenyl)-butyl]ester; (2R)-2-amino-4-[3-(4-cyclohexyloxybutyl)-benzo[b]thien-6-yl]-2-methylbutan-1-ol; the phosphate salt of 2-amino-2-[2-(4-octylphenyl)ethyl]propane-1,3-diol; 2-amino-4-(4-heptyloxyphenyl)-2-methyl-butanol, the hydrochloride salt of 2-amino-4-(4-heptyloxyphenyl)-2-methyl-butanol; the R-enantiomer of 2-amino-4-(4-heptyloxyphenyl)-2-methyl-butanol; the phosphate salt of 2-amino-4-(4-heptyloxyphenyl)-2-methyl-butanol; 2-amino-2-{2-[4-(1-oxo-5-phenylpentyl)phenyl]ethyl}propane-1,3-diol, hydrochloride; or phosphate salt of 2-amino-2-{2-[4-(1-oxo-5-phenylpentyl)phenyl]ethyl}propane-1,3-diol; Rapamycin or Rapamycin derivatives; tacrolimus; Cyclosporins, Cyclosporin “A”, Cyclosporin G, [O-(2-hydroxyethyl)-(D)Ser]⁸-Ciclosporin, [3′-dehydroxy-3′-keto-MeBmt]¹-[Val]²-Ciclosporin; and FK506, or in any case a pharmaceutically accepted salt thereof.

22. Combination according to claim 21, wherein the immunosuppressive or immunomodulator agent is a S1P receptor agonist or, in each case, a pharmaceutically acceptable salt thereof.

23. Combination according to claim 21, wherein the immunosuppressive or immunomodulator agent is a S1P receptor agonist selected from 2-amino-2-[2-(4-octylphenyl)ethyl]propane-1,3-diol, 2-amino-2-[4-(3-benzyloxyphenoxy)-2-chlorophenyl]propyl-1,3-propane-diol or 2-amino-2-[4-(benzyloxyphenylthio)-2-chlorophenyl]propyl-1,3-propane-diol, in free form or, in each case, a pharmaceutically acceptable salt thereof.

24. Combination according to claim 19, wherein vildagliptin or a pharmaceutically acceptable salt thereof, is administered in an amount between 25 and 150 mg or between 50 and 100 mg daily.

25. Combination according to claim 21, wherein 2-amino-2-[2-(4-octylphenyl)ethyl]propane-1,3-diol, or a pharmaceutically acceptable salt thereof, is administered in an amount between 1 mg and 10 mg daily.

26. Combination according to claim 21, wherein 2-amino-2-[2-(4-octylphenyl)ethyl]propane-1,3-diol, chlorhydrate, is administered in an amount between 0.5 mg and 6 mg daily or between 2.5 mg and 5 mg daily.

27. (canceled)

28. (canceled)
